(19) United States
(12) Patent Application Publication
(10) Pub. No.: US 2010/0190686 A1
(43) Pub. Date: Jul. 29, 2010

(54) NOVEL SECRETED PROTEINS OF ADIPOCYTES FOR DIAGNOSTIC PURPOSES

(75) Inventors: Robert L. Wells, Athens, GA (US); Jae-min Lin, Athens, GA (US); Dorothy Hausman, Watkinsville, GA (US)

Correspondence Address: COLEMAN SUDOL SAPONE, P.C., 714 COLORADO AVENUE, BRIDGE PORT, CT, 06605-1601, US

(73) Assignee: The University of Georgia Research Foundation, Athens, GA (US)

(21) Appl. No.: 12/452,061

(22) PCT Filed: Jun. 23, 2008
(86) PCT No.: PCT/US2008/007790

§ 371 Date: Apr. 9, 2010

Related U.S. Application Data
(60) Provisional application No. 60/936,926, filed on Jun. 22, 2007.

(51) Int. Cl. C12Q 1/34; C12Q 1/02; G01N 33/53; A61K 31/175; A61K 31/155; A61K 31/4439; A61K 31/7034; A61K 31/45; A61K 31/195; A61K 31/4985; A61K 38/22; A61K 38/28; A61P 3/10

(52) U.S. Cl. 514/3; 435/18; 435/29; 435/7.92; 435/7.1; 514/592; 514/635; 514/342; 514/42; 514/315; 514/563; 514/249; 514/12

(57) ABSTRACT

The present invention relates to the discovery of polypeptide biomarkers excreted into the blood stream (serum or plasma) and/or urine from adipocytes and their use to determine the existence of metabolic syndrome (prediabetic) and/or diabetic conditions (including type 2 diabetes) including insulin resistance and/or glucose intolerance, their use to monitor the state of metabolic syndrome or diabetes toward a state of control and/or cure of the disease state or condition and their use to monitor the long-term health of the patient by determining the existence of metabolic syndrome (prediabetic conditions insulin resistance and/or glucose intolerance) or the existence of type 2 diabetes and to identify potential antidiabetes agents. Methods of identifying potential agents to be used in the treatment of metabolic syndrome and/or type2 diabetes and assays for assisting in the diagnosis of metabolic syndrome and/or type 2 diabetes are additional aspects of the invention. Lipoprotein lipase; Quiescin Q6; Cathepsin B; Complement Component 6; Hippocampal Cholinergic Neurostimulating Protein (HCNP); Serine Protease Inhibitor 2C; Adiponectin; Angiotensinogen (angiotensin); Cyclophilin A; Laminin B1 subunit 1; cartilage glycoprotein 39; complement factor MASP-3 (MASP-3); Niemann-Pick disease, type C2, isoform CRA b (NPC2); Tetranectin; tissue inhibitor of metalloproteinase 2 (TIMP2); Superoxide dismutase secreted; and Spondin 1

### RELATED APPLICATIONS

This application claims the benefit of priority of provisional patent application Ser. No. U.S. 60/936,926, filed Jun. 22, 2007, which application is incorporated by reference in its entirety herein.

### FIELD OF THE INVENTION

The present invention relates to the discovery of polypeptide biomarkers excreted into the blood stream (serum or plasma) and/or urine from adipocytes and their use to determine the existence of prediabetic (metabolic syndrome) and/or diabetic conditions including insulin resistance and/or glucose intolerance, their use to monitor the state of a prediabetic condition or diabetes toward a state of control and/or cure of the disease state or condition and their use to monitor the long-term health of the patient by determining the existence of prediabetic conditions (insulin resistance and/or glucose intolerance) or the existence of diabetes and to identify potential antidiabetes agents.

In the present invention, secreted proteins from adipocytes (fat) whose expression is changed upon a shift from insulin responsive to insulin resistant (the hallmark of type II diabetes) conditions were elucidated by using a combination of cell culture conditions and high throughput, high sensitivity chromatography and mass spectrometry. As approximately a ¼ of the US adult population is poorly-defined as prediabetic (metabolic syndrome) but only ¼ of these will develop type II diabetes, these markers could be used to diagnose the development of type II diabetes (hopefully, before it is irreversible) as well as the complications that arise from insulin resistance such as heart, kidney, macrovascular, microvascular, and retinal degeneration disease. Furthermore, agonist and or antagonist agents of these secreted proteins could be used as possibly therapies for the previously listed diseases as well as modulating obesity, eating behavior, and inflammation.

### BACKGROUND OF THE INVENTION

Type II diabetes affects more than 7% of the American adult population. Chronic hyperglycemia is the hallmark of all types of diabetes. In type II diabetes, insulin resistance is a primary, early feature and along with “glucose toxicity” is likely responsible for the plethora of observed patient complications such as cardiovascular disease, blindness, and kidney disease (ref). Insulin resistance results in defects in a multitude of systems including pancreatic β-cells, the liver, and peripheral insulin-responsive tissues (adipocytes and skeletal muscle) (ref). Insulin normally regulates a variety of processes such as glucose uptake, glycogen synthesis, triglyceride synthesis, lipolysis, gluconeogenesis, apoptosis, cell growth, differentiation, and adipocytokine (adipokine) secretion.

It has been suggested that insulin resistance is an adaptation to nutrient excess and lack of energy expenditure. Increased flux through the hexosamine biosynthetic pathway (HBP) that converts fructose-6-phosphate (a glycolysis intermediate) to UDP-GlcNAc (a donor sugar nucleotide) has been heavily implicated in the induction of insulin resistance. UDP-GlcNAc serves as a donor for glycosylation including the O-GlcNAc modifcation of nuclear and cytosolic proteins. Elevation in global O-GlcNAc modification of proteins has been directly demonstrated to generate insulin resistance and glucose disposal defects in adipocytes and muscle cells in culture as well as in rodent models.

In the last decade, it has become apparent that adipose tissue, in addition to being an energy storage depot, is an endocrine tissue. In addition to the free fatty acids secreted by adipocytes, a number of adipocyte secreted proteins, adipocytokines, can act in an autocrine, paracrine, or endocrine fashion to modulate a variety of processes including insulin sensitivity, inflammation, obesity, hypertension, food intake (anorexigenic and orexigenic), and general energy homeostasis. Adipocytokine secretion has been implicated in a variety of the complications associated with type II diabetes.

A few groups have sought to define the secreted proteome of adipocytes by a variety of approaches. Most of the studies that focused on differential protein expression compared pre-adipocytes to differentiated adipocytes. Secretion of many adipocytokines in mature adipocytes appears to be regulated by insulin stimulation and glucose levels (ref). In at least two cases, for leptin and adiponectin, transcriptional regulation of the adipocytokines is regulated by the hexosamine biosynthetic pathway. Furthermore, mice with elevated O-GlcNAc levels display hyperleptinemia.

In order to more completely define the repertoire of adipocytokines, we report here a detailed proteomic analysis of the secreted proteome of rodent adipocytes (both immortalized mouse and primary rodent derived) using a combination of shotgun proteomics, glycosylation site mapping, and 2D-reverse phase separations that allowed us to assign more than two hundred secreted polypeptides with high confidence. We also demonstrate via quantitative proteomics that many of the secreted proteins that are regulated by classical induction of insulin resistance (hyperglycemia and chronic insulin exposure) are regulated in a similar fashion by elevated O-GlcNAc level-induced insulin resistance.

There exists a need in the art for a simple, reliable diagnostic test for use in determining the existence of a prediabetic or diabetic condition in a patient in need. There is a further need for a reliable diagnostic test for use in determining whether a prediabetic condition has worsened into a diabetic condition. There is still a further need for a simple, reliable diagnostic test for monitoring the progression of the therapy of diabetes. There is an additional need for a simple, reliable diagnostic test for monitoring the cure/status of a prediabetic (metabolic syndrome) condition or diabetes in a treated patient.

### OBJECTS OF THE INVENTION

It is an object of the invention to provide methods for diagnosing metabolic syndrome and/or type 2 diabetes using simple tests which identify concentrations (amounts) of polypeptide markers of these disease states or conditions.

It is another object of the invention to provide methods for monitoring treatment of metabolic syndrome and/or type 2 diabetes using a simple blood (blood, plasma, serum) or urine test.

It is an additional object of the invention to provide assays which can be used to test for activity as pharmacological agents which may be used to treat metabolic syndrome and/or type 2 diabetes.

Any one or more of these and/or other objects of the invention may be readily gleaned from a careful reading of the description of the invention presented herein.

### BRIEF DESCRIPTION OF THE FIGURES

FIG. 1 shows that O-GlcNAc Levels are elevated under insulin resistant conditions. 3T3-L1 adipocytes (A) or rat primary adipocytes (B) were maintained in an insulin responsive growth condition (low (physiological) glucose, LG) or grown under insulin responsive conditions (either high glucose and chronic insulin exposure (HG+INS) or treated with PUGNAc (PUG)). Equal amounts of proteins from whole-cell extracts were separated by SDS-PAGE and Western blotting performed using the anti-O-GlcNAc antibody RL-2. ERK-2 western blotting was performed as well to demonstrate equal loading.

FIG. 2 shows a schematic diagram of an experimental approach. Adipocytokines are harvested from adipocytes that are insulin responsive (LG, low glucose) or that have been shifted to an insulin resistant state by the addition of PUGNAc to elevate O-GlcNAc levels or by the combination of hyperglycemia (HG) and hyperinsulinemia (INS).

FIG. 3 shows assigning the secreted proteome of adipocytes. Following tryptic digest, the secreted proteome is analyzed by shotgun proteomics and off-line HPLC separation into 5 fractions each followed by LC-MS/MS.

FIG. 4 shows differential secretion of adipocytokines as determined by reconstructed ion chromatograms. A peptide from serine protease inhibitor 2C (A) or tetranectin (B) that was isolated and reconstructed from the secreted proteome of rat adipocytes is shown from two different biological replicates and three different culturing conditions. Reconstructed ion chromatograms for both peptides clearly shown an upregulation when the adipocytes are shifted from insulin responsive conditions (LG, normalized to 1.00) to insulin resistant conditions (HG+INS and LG+PUG).

FIG. 5 shows Table 1, which shows the calculation of false-discovery rates which is described in the examples section of the present application.

FIG. 6 shows Table 2, which lists novel secreted proteins from immortalized mouse adipocytes.

FIG. 7 shows Table 3, which shows mouse adipocytokines regulated by insulin resistance. In the table, shown in (HG+I)/LG and PUG/LG are the ratios of the average unique peptides from each secreted proteome for a particular sample. (L) refers to 3T3-L1, (F) refers to 3T3-F442A; when no peptides were detected under a given condition the total number of unique peptides assigned to the protein are shown in brackets [ ].

FIG. 8 shows Table 4, which shows novel secreted proteins from primary rat adipocytes identified in the examples section.

FIG. 9 shows Table 5, which shows rat adipocytokines regulated by insulin resistance which were identified in the examples section. In the table, shown in (HG+I)/LG and PUG/LG are the ratios of the average number of unique peptides from each condition. (1) refers to LC-MS/MS shotgun analysis and (2) referes to 2D-LC-MS/MS analysis; when no peptides were detected under a given condition the total number of unique peptides assigned to the protein are shown in brackets [ ].

FIG. 10 shows Table 6, which shows the sites of N-linked glycosylation in rodent adipocytokines.

### BRIEF DESCRIPTION OF THE INVENTION

The present invention evaluates polypeptide biomarkers of adipocytes to determine the level of glucose intolerance and/or insulin resistance as well as the existence of a prediabetic (metabolic syndrome) or diabetic condition, including type II diabetes. The present invention provides a method of determining the existence of prediabetes and diabetes (especially type 2 diabetes) and the therapeutic prognosis for that condition and predicting therapeutic outcome of a patient by analyzing the expression of unglycosylated and/or glycosylated polypeptide biomarkers obtained from the serum, plasma or urine of a patient or subject. The method comprises the steps of first establishing the threshold value (a predetermined value) of at least one prognostic polypeptide biomarker of an adipocyte under normal, prediabetic or diabetic condition, preferably at least two prognostic polypeptide biomarkers, at least three prognostic polypeptide biomarkers, at least four prognostic biomarkers, at least five prognostic biomarkers or preferably, at least six prognostic polypeptides (in a human patient), as many as ten, as many as 15, as many as 20, and as many as 22 or more prognostic polypeptides in a human patient or in an animal (veterinary indications). The amount of the prognostic polypeptide(s) from a patient is determined. The amount of the prognostic polypeptide(s) present in that patient's serum is compared with the established threshold (predetermined) value of the prognostic polypeptide biomarker from control sera which is indicative of a prediabetic (metabolic syndrome) or diabetic condition, the prospects of a therapeutic success or failure, the monitoring of treatment, or the existence of continued diabetes in a patient whereby the existence of a condition or outcome of the patient is determined. In many (most) instances, upregulation (i.e., expression of significantly more than a threshold or standard-predetermined value) of one or more prognostic biomarkers is indicative of metabolic syndrome and/or type 2 diabetes. In others, down regulation (i.e., expression of significantly less than a threshold or standard—a predetermined value) is indicative of metabolic syndrome and/or type 2 diabetes.

In certain embodiments, the amount or concentration of the prognostic polypeptide(s) can be based on antibody detection, for example, monoclonal antibody detection or determining and identifying glycosyl residues on biomarker polypeptides using art recognized methods of identifying and measuring same. In a number of embodiments, the evaluating is of at least one prognostic polypeptide, preferably a plurality of prognostic polypeptides, preferably at least three prognostic polypeptides, and more preferably at least five or more polypeptides as otherwise described herein, including as many as 22 or more polypeptides (biomarkers). The prognosis contributes to identification of metabolic syndrome or type 2 diabetes (preferably, type 2 diabetes) and the selection of a therapeutic strategy consistent with the identification of metabolic syndrome or type 2 diabetes, which may be traditional therapy (nutrition and/or exercise), or a more aggressive therapy (antidiabetes medications, medications to address secondary conditions such as hyperlipidemia, hypercholesterolemia, high blood pressure, etc.) based upon a traditional therapy or non-traditional (including experimental) therapy.

The present invention is directed to methods for outcome prediction and risk classification in prediabetic conditions (metabolic syndrome) and diabetes (especially type 2 diabetes). In one embodiment, the invention provides a method for classifying the existence and severity of a prediabetic or diabetic condition (including type 2 diabetes) in a patient that includes obtaining a biological sample from a patient; determining the expression level for a selected polypeptide(s) in the blood, serum, plasma or urine of a patient suspected of being prediabetic or having diabetes, more preferably a group of selected biomarker polypeptides to yield an observed polypeptide(s) production level (observed level); and comparing the observed level for the selected polypeptide(s) to control polypeptide levels. The control level can be the concentration observed for the polypeptide(s) in a control sample (which may be a normal control, a prediabetic/metabolic syndrome control or a diabetic control), or a predetermined level for the polypeptide marker. An observed blood/serum/plasma/urine polypeptide level (generally higher, but in certain instances, lower as indicated further herein) that differs from the control polypeptide level (in that same biological sample) is indicative of a disease or condition classification. In another aspect, the method can include determining a profile for selected polypeptide products in the biological sample to yield an observed polypeptide profile; and comparing the observed polypeptide profile for the selected polypeptide(s) to a control polypeptide profile for the selected polypeptides that correlates with a disease classification, for example type 2 diabetes, metabolic syndrome, insulin resistance, glucose intolerance, among others, wherein a similarity between the observed polypeptide profile and the control polypeptide profile is indicative of the disease or condition classification. Therapeutic or other remedial steps may thereafter be taken for the patient consistent with the diagnosis and the present method may be used to assess the treatment and define a cure or favorable treatment outcome, where relevant.

The disease classification can be, for example, a classification based upon disease state, based on predicted outcome (control or cure of diabetes, increase in glucose tolerance, reduction in insulin resistance); a classification based upon clinical characteristics of patients, a classification based on karyotype; a classification based on precondition or condition subtype; or a classification based on disease etiology.

Where the classification is based upon disease, in particular, the existence of metabolic syndrome or type 2 diabetes, the observed blood, serum, plasma or urine gene product (polypeptide) is preferably a gene product/biomarker selected from at least one, preferably at least two, at least three, at least four, at least five, at least six, at least seven, at least eight at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two or more gene products selected from the following gene products as set forth in Table IA, hereinbelow.

The present invention may also be used to assist in the detection/diagnosis and/or therapy of various conditions or disease states which are found secondary to metabolic syndrome or type 2 diabetes including, for example, obesity, cardiovascular disease including atherosclerosis and hypertension, pancreatic cancer, retinopathy, renal disease, erectile dysfunction, dental disease, ketoacidosis, depression and neuropathy, among a number of others.

In the present method, the existence of expression levels of one or more of the described gene products is predictive of the existence of a condition of metabolic syndrome (e.g., insulin resistance and/or glucose intolerance, etc.) or type 2 diabetes. In the present invention, measuring concentration of gene products/biomarkers will result in assessing the existence and recommended therapy for treating the diagnosed condition or disease state.

In the case of determining whether a therapeutic intervention is providing favorable results (favorable progression) in reducing or and/or curing the prediabetic (metabolic syndrome) or diabetic disease state in the patient, one or more gene products from Table 1A may be used to provide a polypeptide level as indicated above, which may be compared to the expression level of the expressed product in a control tissue sample (which may be normal or not), wherein a level of expression in the sample obtained from blood, serum, plasma or urine of the treated patient which is below or above an elevated control or approaching or equivalent to a normal control, evidences favorable therapeutic progression and/or cure of the condition or disease state. Thus, where the expression level (concentration measured) of a polypeptide marker is above or below a control level (for a control group without metabolic syndrome or type 2 diabetes) when the patient is typically diagnosed with or has metabolic syndrome or type 2 diabetes, the expression level (concentration measured) of the same polypeptide marker which is at a control level or below or above the control level, respectively, is evidence of therapeutic progress or a cure.

In an alternative embodiment, the present invention may be used to determine the existence of metabolic syndrome or a diabetic disease state, by measuring in the blood, serum, plasma or urine of a subject or patient the expression of at least one expression product identified in Table 1A, hereinbelow, wherein the increased or decreased expression (generally, the significant measured expression is at least about 1.1 (110%), 1.2 (120%), 1.3 (130%), 1.4 (140%), preferably at least about 1.5 (150%), at least about 2 times (200%) or at least 3 times (300%) above or below, as appropriate, the baseline of control) of one or more of the expression products as set forth in Table 1A hereof is evidence of the existence of a diabetic condition such as insulin resistance and/or glucose intolerance (metabolic syndrome), or the existence of diabetes (especially type 2 diabetes). Treatment (dietary, exercise, pharmaceutical) intervention as otherwise disclosed herein may thereafter be considered.

Alternatively, the invention may rely on measuring the previous polypeptide markers as indicated within a longer list according to Table 1A of up to 22 or more polypeptide biomarkers as otherwise described herein. Measurement of all 22 biomarkers (or more, for example as otherwise provided in the examples section herein) may also be performed to provide an accurate assessment of the type of tissue (normal, prediabetic or diabetic), whether the condition or disease state is in the early or more advanced stages and the likelihood that the condition or disease state will respond to therapeutic intervention, whether that intervention is nutritional, physical (exercise), pharmaceutical or perhaps even surgical (e.g., gastric bypass surgery).

The invention further provides for a method for predicting therapeutic outcome in a patient that includes obtaining a biological sample from a patient in the form of a blood (or urine) sample; determining the concentration level for selected polypeptide biomarkers associated with outcome in said sample to yield an observed polypeptide level of the selected polypeptide(s); and comparing the observed concentration level for the selected polypeptide(s) in the patient's sample to a control concentration level for the selected polypeptide(s). The control level for the selected polypeptide(s) can include the concentration level for the selected polypeptide(s) observed in a control sample, or a predetermined concentration level for the selected polypeptide(s); wherein an observed level that is different from (significantly above or below) the control level for the selected polypeptide(s) is indicative of favorable (or unfavorable) intervention or therapeutic outcome.

Optionally, the method further comprises determining the concentration level for other polypeptide(s) within the list of polypeptide(s) and comparing in a similar fashion the observed levels for the selected polypeptide(s) with a control level for those polypeptide(s), wherein an observed level for these polypeptide(s) that is different from (above or below) the control level for that polypeptide(s) is further indicative of favorable (or unfavorable) intervention or therapeutic outcome.

The invention further includes a method for treating metabolic syndrome or type 2 diabetes comprising administering to a patient with metabolic syndrome or diabetes a therapeutic agent which is used to treat or alleviate one or of the secondary conditions associated with metabolic syndrome or diabetes or alternative, that modulates the amount or activity of the polypeptide(s) associated with outcome, in particular, one or more of the biomarker proteins which are presented in Table 1 herein. In the present method, the existence of concentration levels of one or more of the polypeptides is predictive of the existence of a prediabetic condition (metabolic syndrome) and/or diabetes and the inhibition (or, in certain instances, the raising of levels, depending upon the polypeptide measured) of any one or more of these polypeptides represents a potential therapeutic target for the prevention (reducing the likelihood) or treatment of diabetes (especially type 2 diabetes). Preferably, the method modulates (inhibits, or in certain instances, raises the levels depending on outcome) at least two of the above polypeptides as set forth above to a level above or below a predetermined expression level. It is noted that further accuracy may be afforded by also measuring a number of other polypeptides selected from the group consisting of those set forth in Table I hereinbelow. Monitoring of therapies which are non-pharmaceutical (nutrition, exercise) are additional aspects of the present invention.

Also provided by the invention is an in vitro method for screening a compound useful for treating prediabetic conditions (metabolic syndrome) and/or preventing (i.e., reducing the likelihood) metabolic syndrome and/or diabetes (especially type II diabetes). This aspect of the invention further provides an in vivo method for evaluating a compound for use in preventing or treating prediabetic conditions and/or diabetes. The candidate compounds are evaluated for their effect on the concentration of one or more polypeptides associated with outcome in diabetes or related conditions as described above, preferably at least two of those polypeptides, more preferably at least three of those polypeptides, four of those polypeptides, five, six, seven, eight, nine, ten, eleven, twelve, thirteen, fourteen, fifteen, sixteen, seventeen, eighteen, nineteen, twenty, tweny-one, twenty-two or more of those gene products as otherwise described herein. Cells may be engineered to overexpress or underexpress any one or more of the gene products associated with metabolic syndrome and/or type 2 diabetes as otherwise described herein using standard techniques available in the art and the engineered cells may be used to identify potential therapeutic agents for metabolic syndrome and/or type 2 diabetes. A compound which is shown to influence the expression of the gene product associated with metabolic syndrome and/or type 2 diabetes (either up or down, as appropriate), the compound may be identified as a potentially therapeutically active compound.

In Table 1A, below is presented the proteins secreted from fat cells that the present inventors identified as being upregulated (overexpressed) or downregulated (underexpressed) as cells are shifted from being insulin responsive (normal, healthy) to insulin resistant (type II diabetic-like). The development of a diagnostic test kit for determining the likelihood of the development of diabetes is an aspect of the present invention. This diagnostic test relates to a multiplexed kit to detect at one, preferably at least two, preferably at least three (3), and preferably more, including 4, 5, 6, 7, 8, 9, 10, 11, 12, 13, 14, 1, 16, 17, 18, 19, 20, 21, 22 or or more of these proteins and other proteins in patient biological samples.

The concept is that these proteins are detected first in the blood, serum, plasms or urine from diabetics and non-diabetics. The retrospective study is based upon blood stored for 20 years, and the method involves using blood samples from diagnosed type 2 diabetics (and matched controls) 6 months, 1 year, 2 year, etc. before diagnosis of type 2 diabetes to see how early one can detect any one or more these proteins. The present invention also relates to a method to detect insulin resistance and/or glucose intolerance and a development towards type 2 diabetes while the condition is still reversible or at least at a point where one can start treating the patient to improve outcome.

The present method relates to measuring the concentration or amount of a polypeptide in biological sample (blood, serum, plasma or urine) taken from a patient and determining whether or not a patient has metabolic syndrome and/or type 2 diabetes from the expression of the polypeptide(s), wherein the polypeptide is selected from the group consisting of at least one polypeptide which is upregulated in patients having metabolic syndrome and/or type 2 diabetes selected from the group consisting of Lipoprotein lipase (LPL); Quiescin Q6; Cathepsin B; Complement Component 6; Hippocampal Cholinergic Neurostimulating Protein (HCNP); Serine Protease Inhibitor 2C; Adiponectin (ACRP30); Angiotensin (Angiotensinogen); Cyclophilin A (Peptidylprolyl isomerase A); Laminin B1 subunit 1 (laminin, beta 1 precursor); GP-39 (chitinase 3-like 1, cartilage glycoprotein 39); MASP-3 (complement factor MASP-3); NPC2 (Niemann-Pick disease, type C2, isoform CRA_b); Tetranectin; TIMP2 (tissue inhibitor of metalloproteinase 2; TIMP-2); Superoxide dismutase, secreted (Superoxide dismutase 3, Extracellular);and Spondin 1 (spondin 1, extracellular matrix protein); or at least one polypeptide polypeptide which is downregulated in patients having metabolic syndrome and/or type 2 diabetes selected from the group consisting of matrix metalloproteinase 2 (MMP2, collagenase, type IV; gelatinase A); Slit homolog 3; Retinoid-inducible serine carboxypeptidase 1 (Serine carboxypeptidase 1); HtrA serine peptidase 1 (HtrA serine peptidase 1 precursor); and Fibrinogen/angiotension related protein; comparing the concentration or amount of the measured polypeptide(s) with a threshold or standard value such that a diagnosis of metabolic syndrome or type 2 diabetes may be made. It is noted that once it is determined from the polypeptide measurement that the patient has metabolic syndrome or type 2 diabetes, further diagnosis consistent with a diagnosis of metabolic syndrome or type 2 diabetes may be made.

In various aspects of the method of the present invention at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two of the above-referenced polypeptides are measured and a diagnosis is made based upon the upregulation or downregulation of the polypeptide(s) as measured and compared with a standard or threshold value. Metabolic syndrome may be diagnosed if the assay measures any number of polypeptides as described above and all of the polypeptides exhibit upregulation or downregulation compared to a threshold or standard value which is consistent with metabolic syndrome or type 2 diabetes. In addition, in the present invention, when at least four polypeptides described above are measured and wherein at least half of the measured polypeptides exhibit upregulation or downregulation (as appropriate) when compared to a threshold or standard measurement, a diagnosis of metabolic syndrome or type 2 diabetes may be made. Once a diagnosis of metabolic syndrome and/or type 2 diabetes is made; appropriate therapeutic intervention is then recommended, as otherwise described herein.

In preferred aspects of the present invention the method (preferably, as a diagnostic assay) measures at least one (upregulated) polypeptide in a biological sample taken from a patient to be diagnosed wherein the polypeptide is selected from at least one polypeptide from the group consisting of Lipoprotein lipase (LPL); Quiescin Q6; Cathepsin B; Complement Component 6; Hippocampal Cholinergic Neurostimulating Protein (HCNP); and Serine Protease Inhibitor 2C; comparing the measurement of the polypeptide(s) in the patient's biological sample with a threshold or standard value and diagnosing metabolic syndrome or type 2 diabetes if a polypeptide is upregulated compared with a threshold or standard. In aspects of the invention, one, two, three, four five and six polypeptides of the above polypeptides are measured, compared with a threshold or standard and a diagnosis of the patient is made when all the polypeptides measured exhibit upregulation compared to the threshold or standard value. In certain aspects of the invention, at least four of the above-identified polypeptides are measured and a diagnosis of metabolic syndrome is made if at least half of the measured polypeptides are upregulated when compared with the threshold or standard.

Although any one or more polypeptides of the twenty-two polypeptides which are described as biomarkers may be measured for diagnostic value in the present invention, in certain preferred aspects of the invention, at least one, two, three, four, five or six polypeptide(s) selected from the group consisting of Lipoprotein lipase (LPL); Quiescin Q6; Cathepsin B; Complement Component 6; Hippocampal Cholinergic Neurostimulating Protein (HCNP); and Serine Protease Inhibitor 2C (SPARC) is measured with at least one additional polypeptide marker of the remaining sixteen polypeptides (of the twenty-two polypeptides) listed.

It is noted that as more polypeptide markers are measured and compared to threshold or standard values, and determined to be upregulated or downregulated (as indicative of metabolic syndrome or type 2 diabetes, the reliability of the diagnosis increases.

The method of the present invention may be used to diagnose metabolic syndrome and/or type 2 diabetes and conditions which are indicative of the syndrome including insulin resistance and glucose intolerance, among others including dyslipidemia, hypercholesterolemia, high blood pressure and risk of cardiovascular disease. The present method may therefore provide information to a patient to change diet and/or lifestyle, to accept pharmacological intervention. In addition to diagnosing metabolic syndrome and/or type 2 diabetes, the present method may also be used to determine whether therapeutic intervention is having a favorable influence on the condition or disease state diagnosed or whether the patient no longer has the diagnosis because there has been a cure.

### DETAILED DESCRIPTION OF THE INVENTION

Polypeptide marker profiling can provide insights into disease etiology and genetic progression, and can also provide tools for more comprehensive molecular diagnosis and therapeutic targeting. In addition, the invention has identified numerous polypeptide markers, that are, alone or in combination, strongly predictive of therapeutic outcome in metabolic syndrome or type 2 diabetes. The polypeptides identified herein, can be used to refine risk classification and diagnostics, to make outcome predictions and improve prognostics, and to serve as therapeutic targets in identifying novel agents which may find pharmacological utility in treatment metabolic syndrome and/or type 2 diabetes.

The following terms shall be used to describe the present invention. It is noted that in describing the present invention, in cases where a term is not specifically defined herein, that term is given a definition which is consistent with its use in context as determined by one of ordinary skill in the art.

Unless otherwise specified, “a,” “an,” “the,” and “at least one” are used interchangeably and mean one or more than one.

The term “patient” is used throughout the specification to describe an animal, generally a mammal such as a domesticated animal and preferably a human, to whom diagnosis of a condition such as metabolic syndrome and/or type II diabetes and/or treatment of such a condition or disease state, including prophylactic treatment, with the compositions according to the present invention is provided. For diagnosis or treatment of those conditions or disease states which are specific for a specific animal such as a human patient, the term patient refers to that specific animal. While the use of the present invention to treat humans represents a primary aspect of the invention, veterinary applications are also intended.

The term “effective” is used herein, unless otherwise indicated, to describe an amount of a compound, composition or component which, when used in context, produces or effects an intended result, whether that result relates to the diagnosis, prevention (reducing the likelihood of) and/or treatment of metabolic syndrome and/or type II diabetes. This term subsumes all other effective amount or effective concentration terms which are otherwise described in the present application.

The term “metabolic syndrome” or “prediabetes” is used throughout the description of the present invention to describe a constellation of conditions that place people at high risk for coronary artery disease. These conditions include type 2 diabetes, insulin resistance, glucose intolerance, obesity, high blood pressure, and a poor lipid profile with elevated LDL (“bad”) cholesterol, low HDL (“good”) cholesterol, elevated triglycerides. All of these conditions are associated with high blood insulin levels. The fundamental defect in the metabolic syndrome is insulin resistance in both adipose tissue and muscle. Drugs that decrease insulin resistance also usually lower blood pressure and improve the lipid profile. KD3010, an orally administered PPARδ selective agonist, may be effective in this regard. In addition, diet may improve insulin resistance and/or glucose intolerance a variety of omega-3 fatty acids including (EPA, DHA from fish), conjugated linoleic acid (dairy) and alpha-linolenic acid (flaxseed) have been shown to have an impact on insulin resistance.

Metabolic syndrome is estimated to be affecting 16 million or more people (that is in addition to the estimated 17 million people with diabetes). While the name is new, the condition certainly is not. Metabolic syndrome was originally referred to as impaired glucose tolerance or impaired fasting glucose. By definition, prediabetes is a condition where one has fasting blood sugar levels above normal (blood sugar between 100-125 mg/dl) but the blood sugar levels are not high enough to diagnose diabetes (fasting blood sugar above 126 mg/dl).

Prediabetes or diabetes risk increases with the following factors:
family history of diabetes,
non-European and non-Asian ethnicity,
overweight or obese,
high blood pressure
low HDL cholesterol and high triglycerides
history of gestational diabetes (diabetes during pregnancy) or having given birth to a baby weighing more than 9 lbs

Those who are at risk are prime candidates for the present invention. A diagnosis of metabolic syndrome or impaired glucose tolerance can be met with action to prevent or delay the onset of diabetes.

Those with metabolic syndrome or prediabetes are more likely to be diagnosed with Type 2 diabetes in the future, but there are preventative measures. Following a diet low in fat and a reduction in calories, physical exercise, and weight loss if overweight can help prevent or delay diabetes. These preventative measures can “turn back the clock” and return elevated blood sugars to normal levels.

Therapeutic intervention in patients diagnosed with metabolic syndrome (as well as type 2 diabetes) may include therapeutic lifestyle changes (healthful eating, weight loss, increased exercise), treatment of dyslipidemia with statins, nicotinic acid and/or bile acid sequestrants (fibrates, including gemfibrozil, fenofibrate), treatment of insulin resistance with metformin, the thiazolidinediones, including troglitazone, pioglitazone and rosiglitazone and the treatment of hypertension with ACE inhibitors, such as ramipril, and angiotensin receptor blockers (ARBs), such as losartan. Other therapeutic modalities will be used consistent with the disease state or condition manifest in the patient diagnosed with metabolic syndrome. Many, if not all, of the therapeutic approaches which are used to treat metabolic syndrome are often effective in treating type 2 diabetes.

Metabolic syndrome is also referred to as insulin resistance syndrome, syndrome X, dysmetabolic syndrome X, and Reaven syndrome. The syndrome was first described at the 1988 Banting Lecture of the annual meeting of the American Diabetes Association. It generally represents a condition which often is, but is not necessarily a pre-condition to type II diabetes. Approximately 25% of patients with metabolic syndrome eventually receive a diagnosis of type II diabetes.

The terms “diabetes mellitus type2” “type II diabetes” “noninsulin dependent diabetes melittis” “NIDDM” and “diabetes type 2” are used interchangeably to describe a life-long disease marked by high levels of sugar in the blood. It occurs when the body does not respond correctly to insulin, a hormone released by the pancreas. Type 2 diabetes is the most common form of diabetes and often occurs after a period during which metabolic syndrome is found in a patient.

Diabetes is caused by a problem in the way the body makes or uses insulin. Insulin is needed to move glucose (blood sugar) into cells, where it is used for energy. If glucose does not get into the cells, the body cannot use it for energy. Too much glucose will then remain in the blood, causing the symptoms of diabetes. There are several types of diabetes; the present invention deals with the diagnosis and treatment of type II diabetes, which is usually accompanied by obesity and insulin resistance. “Insulin resistance” refers to a condition within which the insulin produced by the pancreas of a patient cannot get inside fat and muscle cells to produce energy. Since the cells are not getting the insulin they need, the pancreas produces more and more. Over time, abnormally high levels of sugar build up in the blood. This is called hyperglycemia. Many people with insulin resistance have hyperglycemia and high blood insulin levels at the same time. People who are overweight have a higher risk of insulin resistance, because fat interferes with the body's ability to use insulin.

Type II diabetes usually occurs gradually. Most people with the disease are overweight at the time of diagnosis. However, Type II diabetes can also develop in those who are thin, especially the elderly. Family history and genetics play a large role in Type II diabetes. Low activity level, poor diet, and excess body weight (especially around the waist) significantly increase your risk for Type II diabetes.

Other risk factors include:
Race/ethnicity
Presence of metabolic syndrome
Age greater than 45 years
Previously identified impaired glucose tolerance by your doctor
High blood pressure
HDL cholesterol of less than 35 mg/dL or triglyceride level of greater than 250 mg/dL
History of gestational diabetes.

About 90-95% of all North American cases of diabetes are type 2, and about 20% of the population over the age of 65 has diabetes mellitus type 2. The fraction of type 2 diabetics in other parts of the world varies substantially, almost certainly for environmental and lifestyle reasons, though these are not known in detail. Diabets affects over 150 million people worldwide and this number is expected to double by 2025. There is also a strong inheritable genetic connection in type 2 diabetes: having relatives (especially first degree) with type 2 diabetes increases risks of developing type 2 diabetes very substantially. In addition, there is also a mutation to the Islet Amyloid Polypeptide gene that results in an earlier onset, more severe, form of diabetes. About 55 percent of type 2 diabetics are obese—chronic obesity leads to increased insulin resistance that can develop into diabetes, most likely because adipose tissue (especially that in the abdomen around internal organs) is a (recently identified) source of several chemical signals to other tissues (hormones and cytokines). Other research shows that type 2 diabetes causes obesity as an effect of the changes in metabolism and other deranged cell behavior attendant on insulin resistance. Type 2 diabetes is often associated with obesity, hypertension, elevated cholesterol (combined hyperlipidemia), and with the condition often termed metabolic syndrome (also known as Syndrome X, Reavan's syndrome, or CHAOS). In many instances type 2 diabetes is considered an advanced stage of metabolic syndrome. Type 2 diabetes is also associated with acromegaly, Cushing's syndrome and a number of other endocrinological disorders. Additional factors found to increase risk of type 2 diabetes include aging, high-fat diets (especially high saturated fat diets) and a less active lifestyle.

Unlike Type 1 diabetes, there is little tendency toward ketoacidosis in Type 2 diabetes, though it is not unknown. One effect that can occur is nonketonic hyperglycemia. Complex and multifactorial metabolic changes lead to damage and function impairment of many organs, most importantly the cardiovascular system in both types. This leads to substantially increased morbidity and mortality in both Type 1 and Type 2 patients, but the two have quite different origins and treatments despite the similarity in complications.

Genetic factors, usually polygenic, are present in most patients. However, environmental factors such as obesity, lack of exercise and a sedentary lifestyle are thought by most observers to lead to insulin resistance. Certainly not all type 2 diabetics have a family history of the condition.

Insulin resistance means that body cells do not respond appropriately when insulin is present. Other important contributing factors:
increased hepatic glucose production (e.g., from glycogen degradation), especially at inappropriate times
decreased insulin-mediated glucose transport in (primarily) muscle and adipose tissues (receptor and post-receptor defects)
impaired beta-cell function—loss of early phase of insulin release in response to hyperglycemic stimuli
Cancer survivors who received allogenic Hematopoeitic Cell Transplantation (HCT) are 3.65 times more likely to report type 2 diabetes than their siblings. Total body irradiation (TBI) is also associated with a higher risk of developing diabetes.

Type 2 diabetes is a more complex problem than type 1, but is sometimes easier to treat, especially in the initial years when insulin is often still being produced internally. Type 2 may go unnoticed for years in a patient before diagnosis, since the symptoms are typically milder (no ketoacidosis) and can be sporadic. However, severe complications can result from unnoticed type 2 diabetes, including renal failure, blindness, wounds that fail to heal, and coronary artery disease. The onset of the disease is most common in middle age and later life.

Type 2 diabetes (diabetes mellitus type 2) is presently of unknown etiology (i.e., origin). Diabetes mellitus with a known etiology, such as secondary to other diseases, known gene defects, trauma or surgery, or the effects of drugs, is more appropriately called secondary diabetes mellitus. Examples include diabetes mellitus caused by hemochromatosis, pancreatic insufficiency, or certain types of medications (e.g. long-term steroid use). About 90-95% of all North American cases of diabetes are type 2, and about 20% of the population over the age of 65 has diabetes mellitus type 2. The fraction of type 2 diabetics in other parts of the world varies substantially, almost certainly for environmental and lifestyle reasons, though these are not known in detail. There is also a strong inheritable genetic connection in type 2 diabetes: having relatives (especially first degree) with type 2 is a considerable risk factor for developing type 2 diabetes. About 55 percent of type 2 are obese—chronic obesity leads to increased insulin resistance that can develop into diabetes, most likely because adipose tissue is a (recently identified) source of chemical signals (hormones and cytokines). Other research shows that type 2 diabetes causes obesity.

Diabetes mellitus type 2 is often associated with obesity and hypertension and elevated cholesterol (combined hyperlipidemia), and with the condition Metabolic syndrome (also known as Syndrome X, Reavan's syndrome, or CHAOS). It is also associated with acromegaly, Cushing's syndrome and a number of other endocrinological disorders.

Diabetes mellitus type 2 is a chronic, progressive disease that cannot now be cured. There are two main goals of treatment of the disease:
1. reduction of mortality and concomitant morbidity (from assorted diabetic complications)
2. preservation of quality of life

The first goal can be achieved through close glycemic control (i.e., blood glucose levels); the reduction effect in diabetic complications has been well demonstrated in several extensive clinical trials and is thus well established. The second goal is often addressed (in developed countries) by support and care from teams of diabetic health workers (physician. PA, nurse, dietitian or a certified diabetic educator). Knowledgeable patient participation is vital and so patient education is a crucial aspect of this effort.

Type 2 diabetes is initially treated by adjustment in diet and exercise, and by weight loss, especially in obese patients. The amount of weight loss which improves the clinical picture is sometimes modest (5-10 lb); this is almost certainly due to currently poorly understood aspects of fat tissue chemical signalling (especially in visceral fat tissue in and around abdominal organs). In many cases, such initial efforts can substantially restore insulin sensitivity. Therapeutic intervention in patients diagnosed with type 2 diabetes includes dietary intervention (including changing the type of fats ingested to increase omega 3 fatty acid intake-docosohexenoic acid (DHA), eicosopentenoic acid (EPA), conjugated linoleic acid (CLA) and alpha linolenic acid (ALA)), and lifestyle changes, including increasing or adhering to a daily exercise regimen.

Alternatively, drug therapy may be used to treat type 2 diabetes. This involves treatment of the individual with an antidiabetic drugs (all are oral agents “OA”s, with the exception of the GLP analogues, which are injected). The initial choice of anti-diabetic drug has been compared in a randomized controlled trial which found cumulative incidence of monotherapy failure at 5 years of 15% with rosiglitazone, 21% with metformin, and 34% with glyburide. Rosiglitazone had more weight gain and edema. Rosiglitazone may increase risk of death from cardiovascular causes. Pioglitazone and rosiglitazone may increase the risk of fractures.

Antidiabetic drugs include:
Sulfonylureas
Biguanides include metformin and phenformin.
Thiazolidinediones (TZDs) include rosiglitazone, pioglitazone, and troglitazone.
α-glucosidase inhibitors include acarbose and miglitol.
Meglitinides include nateglinide, repaglinide, and their analogs.
Peptide analogs
   Incretin mimetics insulin secretagogues.
   Glucagon-like peptide (GLP) analogs (subcutaneous administration)
   exenatide
   liraglutide (not FDA approved)
   Gastric inhibitory peptide (GIP) analogs
   None are FDA approved
   Incretin enhancers
   sitagliptin
   Amylin agonist analog (slows gastric emptying and suppresses glucagon)
   pramlintide
   Insulin preparations

Preferred pharmacological intervention may include the administration of sulfonylurea agents, including tolbutamide and chlorpropamide; biguanides such as metformin; alpha-glucosidase inhibitors, such as acarbose (Precose) and miglitol (Glyset); the thiazolidinediones, such as troglitazone, pioglitazone and rosiglitazone (insulin sensitizers); and the non-sulfonylurea meglitinides, such as repaglinide (Prandin) or mixtures thereof are useful.

In addition, as in the case of metabolic syndrome, treatment of dyslipidemia with statins, nicotinic acid and/or bile acid sequestrants (fibrates, including gemfibrozil, fenofibrate), treatment of insulin resistance with metformin, the thiazolidinediones (described above), and the treatment of hypertension with ACE inhibitors, such as ramipril, and angiotensin receptor blockers (ARBs), such as losartan may be advantageously used for therapeutic intervention in type 2 diabetes.

The term “polypeptide expression profile” as used herein is defined as the expression level of two or more polypeptide biomarkers. The term polypeptide includes all natural variants (including glycosylated variants) of the polypeptide. These are obtained from a biological sample (blood, serum, plasma or urine) of a patient to be tested and diagnosed.

The term “traditional therapy” relates to therapy (protocol) which is typically used to treat metabolic syndrome (prediabetes) or type 2 diabetes and includes all therapies which are well-known in the art. In the present invention the term “more aggressive therapy” or “alternative therapy” usually means a more aggressive version of conventional therapy typically used to treat prediabetes or diabetes, using exercise, diet, surgery or conventional or traditional pharmaceutical agents at dosages and/or for periods of time in order to increase the likelihood of a favorable therapeutic outcome. It may also refer, in context, to experimental therapies for treating diabetes, rather than simply more aggressive versions of conventional (traditional) therapy.

The term “polypeptides” or “polypeptide biomarkers” refer to the polypeptides which are measured in the method of the present invention and include all glycosylated variants of the unglycosylated polypeptide otherwise described herein. The following table 1 sets forth those polypeptide markers which may be used to identify metabolic syndrome and/or type 2 diabetes

TABLE 1A
Relevant Proteins Expressed By Human Adipocytes

| | Reference |
|---|---|
| Upregulated Polypeptides | Number* |
| Lipoprotein Lipase (LPL) | GI:15030193 |
| Quiescin Q6 Sulhydryl Oxidase 1 | GI:13325075 |
| Cathepsin B | GI:4503139 |
| Complement Component 6 | GI:169636415 |
| Hippocampal Cholinergic Neurostimulating | GI:1352726 |
| Protein (HCNP) or Phosphaticylethanolamine | |
| Binding Protein (PEBP) | |
| Serine Protease Inhibitor 2C (SPARC) | GI:119601994 |
| Adiponectin (ACRP30) | GI:64654883 |
| Angiotensinogen (Angiotensin) | GI:532198 |
| Cyclophilin A (Peptidylprolyl isomerase A) | GI:49522214 |
| Laminin B1 subunit 1 (laminin, beta 1 precursor) | GI:167614504 |
| GP-39 (chitinase 3-like 1, cartilage glycoprotein 39) | GI:119611874 |
| MASP-3 (complement factor MASP-3) | GI:15088517 |
| NPC2 (Niemann-Pick disease, type C2, isoform CRA_b) | GI:119601584 |
| Tetranectin | GI:825722 |
| TIMP2 (tissue inhibitor of metalloproteinase 2; TIMP-2) | GI:298202 |
| Superoxide dismutase, secreted (Superoxide dismutase 3, | GI:15680150 |
| Spondin 1 (spondin 1, extracellular matrix protein) | GI:110347423 |
| | Reference/ |
| Downregulated Polypeptides | Accession Number* |
| MMP2 (collagenase, type IV) matrix | GI:51831776 |
| metalloproteinase 2 (gelatinase A) | |
| Slit homolog 3 | GI:11321571 |
| Retinoid-inducible serine carboxypeptidase 1 | GI:41690765 |
| (Serine carboxypeptidase 1) | |
| Protease, Serine, 11, HtrA serine peptidase 1 | GI:4506141 |
| Fibrinogen/angiotension related protein | GI:21536398 |

*GI reference numbers (search number) provide access to the underlying protein reference at the internet site: ncbi.nlm.nih.gov/sites/entrez?db=Protein

These polypeptides may be glycosylated or unglycosylated and in preferred aspects both glycosylated and unglycosylated are measured. In these instances, a monoclonal antibody binds to an epitope of the polypeptide which occurs in both the glycosylated and unglycosylated polypeptides. Alternatively, monoclonal antibodies may be raised against glycosylated or unglycosylated polypeptides. Analysis of polypeptides (generally, by measuring a concentration of one or more of the above-referenced polypeptides in a patient's blood, serum or plasma, although a urine sample may also be used) according to the present invention may be made through one the following methods.

A direct assay, such as an immunoassay using antibodies that recognize the unglycosylated or glycosylated polypeptide biomarker, is preferred, but other exemplary assays can involve lectins that assay for carbohydrate moieties or any other fingerprinting technique including qualitative or quantitative carbohydrate composition analysis, chromatography, chemical or electrophoresis or isoelectric focusing tests, among others, or any other methods that detect glycosylation variants of polypeptides and/or antibodies to glycosylated or carbohydrate-variant polypeptides. Such assays are described and known in the art. A number of commercially available monoclonal antibodies for the individual polypeptides which are set forth may be used in bioassays, including colorimetric or other bioassays for identifying the concentration/amount of one or more of the polypeptide markers as set forth above in a biological sample taken from the patient.

Immunoassays that can be used to detect unglycosylated and glycosylated polypeptides include, but are not limited to, assays employing specific antibodies to the individual polypeptide biomarkers as otherwise described herein and assays employing nonspecifically defined antibodies obtained by injection of a polypeptide marker obtained from the serum of a patient with an ovarian tumor and/or cancer as described herein, into test animals using standard methods. Many of the monoclonal antibodies which may be used in bioassays which may be used in the present invention are available commercially. Alternatively, antibodies to these polypeptides can be generated by standard means as described, for example, in “Antibodies: A Laboratory Manual” by Harlow and Lane (Cold Spring Harbor Press, 1988), which is hereby incorporated by reference.

For example, a monoclonal anti-polypeptide antibody can be generated by immunizing a mouse with an isolated or recombinant polypeptide (glycosylated or non-glycosylated) pre-treated with neuraminidase, or a cell expressing recombinant the polypeptide pre-treated with neuraminidase. Once an immune response is detected, e.g., antibodies specific for that polypeptide are detected in the mouse serum, the mouse spleen is harvested and splenocytes are isolated. The splenocytes are then fused by well-known techniques to any suitable myeloma cells, for example, cells from cell line SP20 available from the American Type Culture Collection (ATCC). Hybridomas are selected and cloned by limited dilution. The hybridoma clones are then assayed by methods known in the art for cells that secrete antibodies capable of binding the relevant biomarker polypeptide. Ascites fluid, which generally contains high levels of antibodies, can be generated by immunizing mice with positive hybridoma clones.

Any type of fusion phage, monoclonal, or polyclonal antibodies can be used in immunoassays of the invention, so long as the antibodies can be used in a reproducible fashion to identify and quantify polypeptide biomarkers in normal and variant (with cancer) populations.

In one embodiment, an amount of a polypeptide marker (glycosylated or non-glycosylated) can be measured using a capture antibody followed by a labeled secondary antibody using a strategy which is well known in the art. In providing this assay, a first antibody may bind to a polypeptide with polypeptide specificity and a second labeled antibody is used to quantify the target polypeptide. The label on the secondary antibody can comprise any chemical, radioactive, lanthanide, colored dye, or genetic tag used in enzyme-linked immunosorbent assays (ELISAs), Western blots, and other sensitive and specific immunoassays and immunoradiometric assays using known methodology. These include conjugating the antibody with horseradish peroxidase or alkaline phosphatase that are easily measurable, typically using colorimetric, fluorometric or luminescent substrates. Genetic labels include firefly luciferase, employed because luciferase produces a bioluminescent molecule when incubated with its substrate, luciferin. Multiplex kits preferably could be used to detect multiple proteins. These are well known in the art.

In other embodiments, polypeptide-specific antibody may be used as a capture antibody, and an antibody specific to a glycosylated or carbohydrate-variant of the polypeptide and/or an abnormal carbohydrate portion thereof can be used as the secondary labeled antibody in an immunoassay such as those described above. Competitive immunoassays employing antibodies to specific glycosylated polypeptide markers can also be employed to detect and quantify the glycosylated polypeptides.

Alternate embodiments using Phaseoulus Vuglaris Leucoagglutinin (PHA-L) or other carbohydrate-specific lectins can be used in place of the capture antibody or labeled antibody. Alternatively, prior to an immunoassay, a lectin or chromatographic method can be used to extract a glycosylated polypeptide biomarker from a biological sample (serum) or from a fraction that was separated or pooled according to another characteristic, such as pI range (isoelectric focusing). These methods are all well known in the art.

Carbohydrate analyses include qualitative observations of differences in physical properties between non-glycosylated and glycosylated proteins, carbohydrate identification using plant lectins specific to the variant carbohydrate portion of the biomarker (where relevant) by standard lectin screening methods, or any other fingerprinting technique including qualitative or quantitative carbohydrate composition analyses.

### Diagnosis, Prognosis and Risk Classification

Current parameters used for diagnosis, prognosis and risk classification in prediabetes and diabetes are related to clinical data, cytogenetics and response to treatment. They include age, weight, glucose levels, etc. As noted above however, these parameters are not always well correlated with outcome, nor are they precisely predictive at diagnosis.

Prognosis is typically recognized as a forecast of the probable course and outcome of a disease. As such, it involves inputs of both statistical probability, requiring numbers of samples, and outcome data. In the present invention, outcome data is utilized in the form of a favorable outcome and cure. The object is to maintain the patient in a non-diabetic condition, restore glucose tolerance and minimize or reverse insulin resistance.

The ability to determine which cases of metabolic syndrome (prediabetes) and type 2 diabetes will respond to treatment, and to which type of treatment, would be useful in appropriate allocation of treatment resources. It would also provide guidance as to the aggressiveness of therapy in producing a favorable outcome. As indicated above, the various therapies have significantly different risks and potential side effects, especially therapies which are more aggressive or even experimental in nature. Accurate prognosis would also minimize application of treatment regimens which have low likelihood of success and would allow a more efficient aggressive or even an experimental protocol to be used without wasting effort on therapies unlikely to produce a favorable therapeutic outcome, preferably a continuous complete remission. Such also could avoid delay of the application of alternative treatments which may have higher likelihoods of success for a particular presented case. Thus, the ability to evaluate individual cases for markers which subset into responsive and non-responsive groups for particular treatments is very useful.

Current models of metabolic syndrome (prediabetes) and diabetes are poor at distinguishing between conditions which have similar biochemical features but which may vary in clinical course and outcome. Identification of novel prognostic molecular markers is a priority if radical treatment is to be offered on a more selective basis to those patients with disease states which do not respond favorably to conventional therapy (especially nutrition/diet and exercise). A novel strategy is described to discover/assess/measure molecular markers for prediabetes and diabetes by assessing the concentration of polypeptide (including glycosylated polypeptide) biomarkers in patients suspected of being at risk for or have metabolic syndrome or type 2 diabetes and modeling these data based on a predetermined concentration of polypeptide biomarkers for numerous patients having a known clinical outcome. The invention herein is directed to defining different forms and severity of prediabetes and diabetes by measuring the existence and concentration of polypeptide markers which can translate directly into therapeutic prognosis. Such prognosis allows for application of a treatment regimen having a greater statistical likelihood of cost effective treatments and minimization of negative side effects from the different/various treatment options.

In preferred aspects, the present invention provides an improved method for identifying and perhaps classifying metabolic syndrome (prediabetes) and diabetes. Polypeptide marker levels in blood, serum, plasma and/or urine are determined for one or more polypeptides associated with outcome, risk assessment or classification or subtype. Polypeptide markers that are particularly relevant for diagnosis, prognosis and risk classification, especially for type 2 diabetes, according to the invention include those described in the above and in Table 1 herein. The polypeptide marker levels for the product of interest in a biological sample (preferably, serum) from a patient diagnosed with or suspected of having metabolic syndrome or type 2 diabetes are compared to biomarker levels observed for a control sample, or with a predetermined polypeptide biomarker level. Observed levels that are higher or lower than the levels observed for the biomarkers of interest (as defined in table 1) in the control sample or that are higher or lower than the predetermined levels for the biomarkers of interest provide information about the ovarian tumor that facilitates diagnosis, prognosis, and/or risk classification and can aid in treatment decisions, especially whether to use a more of less aggressive therapeutic regimen or perhaps even an experimental therapy. When the levels of multiple polypeptide biomarkers are assessed for a single biological sample, a polypeptide marker profile is produced.

In one aspect, the invention provides serum polypeptide marker profiles that are correlated with outcome (i.e., reduction in symptomology vs. outright cure in prediabetic conditions and/or in diabetes type 2. Assessment of one or more of these polypeptide markers according to the invention, preferably at least two, three, four, five, or more as otherwise described herein (Table 1), or as many as 22 or more polypeptide markers as otherwise described herein in a given gene profile can be integrated into revised risk classification schemes, therapeutic targeting and clinical trial design. In one embodiment, the concentration of particular polypeptide biomarker(s) are measured, and that measurement is used, either alone or with other parameters, to assign the patient to a particular risk category (including a level of metabolic syndrome or type 2 diabetes). The invention identifies several polypeptide biomarkers whose concentration in a patient's serum, either alone or in combination, are associated with subtype and in certain instances therapeutic outcome.

Some of these polypeptide markers exhibit a positive association between concentration level and type of prediabetic or diabetic condition and separately, outcome. For these polypeptide markers, concentration levels (generally in serum) above a predetermined threshold level (or higher than that exhibited by a control sample) is predictive of a given tissue subtype (normal, benign or cancerous), positive outcome (continuous complete remission). Our data suggests that measurement of the concentration levels of at least one of these polypeptide markers, preferably at least two, three, four or five of these polypeptide markers, etc (6,7,8,9,10,11,12,13,14,15,16,17,18,19,20,21 or 22 or more of these polypeptide markers, can be used in refining risk classification and outcome prediction in prediabetic conditions and diabetes. In particular, it is expected such measurements can be used to refine risk classification in patients who exhibit features of prediabetes or diabetes and who could be cured with more intensive or less intensive therapies.

Each of the polypeptide biomarkers may be measured individually or in groups. In preferred aspects the expression at least two, three, four, five, six or more polypeptides in the serum, plasma or urine are measured. Concentration levels for multiple polypeptide biomarkers can be measured (multiplex assays). In certain aspects of the invention where very high reliability is desired/required, then of all 22 polypeptides (as per Table 1) may be measured and compared with a predetermined value such that a measurement above or below the predetermined value of expression for the group of polypeptides which is indicative of a favorable therapeutic outcome or a therapeutic failure which would require more intensive therapy. In the event of a predictive favorable therapeutic outcome, conventional therapy may be used and in the event of a predictive unfavorable outcome (failure), more aggressive therapy may be recommended and implemented.

The expression levels of multiple (two or more, preferably three, four or five, six or more, preferably up to 22 or more polypeptides/gene products as described hereinabove within the polypeptides listed in Table 1A) in one or more lists of genes/polypeptide markers associated with outcome can be measured, and those measurements are used, either alone or with other parameters, to assign the patient to a particular risk category as it relates to a disease state, the existence of diabetes or a predicted therapeutic outcome. For example, concentrations of polypeptide biomarkers can be measured for a patient as described hereinabove using an immunoassay or a related method as otherwise described herein. If the serum polypeptide profile of the patient is similar to that of the list of polypeptides associated with outcome, then the patient can be assigned to a low (or high, as the case may be) risk category. The correlation between polypeptide concentrations and class distinction can be determined using a variety of methods. The information provided by the present invention, alone or in conjunction with other test results, aids in sample classification and diagnosis of disease.

Computational analysis using the polypeptide lists and other data, such as measures of statistical significance, as described herein is readily performed on a computer. The invention should therefore be understood to encompass machine readable media comprising any of the data, including polypeptide lists, described herein. The invention further includes an apparatus that includes a computer comprising such data and an output device such as a monitor or printer for evaluating the results of computational analysis performed using such data.

In another aspect, the invention provides polypeptide profiles that may be correlated with cytogenetics. This allows discrimination among the various karyotypes, which are useful in risk assessment and outcome prediction.

In yet another aspect, the invention provides polypeptide profiles that are correlated with intrinsic disease biology and/or etiology. In other words, polypeptide biomarker profiles that are common or shared among individual diabetes cases in different patients may be used to define intrinsically related groups based upon a biomarker profile that cannot be appreciated or diagnosed using standard means such as morphology, immunophenotype, or cytogenetics.

Some polypeptides in these clusters may be metabolically related, suggesting that a metabolic pathway that is associated with diabetes initiation or progression, etc., thus potentially serving as therapeutic targets.

In yet another aspect, the invention provides polypeptide profiles which may be used to discriminate patient subtypes as otherwise described herein.

It should be appreciated that while the present invention is described primarily in terms of human disease, it is useful for diagnostic and prognostic applications in other mammals as well, particularly in veterinary applications such as those related to the treatment of acute cancers in cats, dogs, cows, pigs, horses and rabbits.

Further, the invention provides methods for computational and statistical methods for identifying polypeptide biomarkers, lists and profiles of polypeptide biomarkers associated with outcome, karyotype, disease subtype and the like as described herein.

In sum, the present invention has identified a group of polypeptide biomarkers which strongly correlate with diagnosis of prediabetic, diabetic and normal cells (adipocytes) as well as favorable/unfavorable outcome in treating prediabetic or diabetic conditions and/or disease states.

### Measurement of Polypeptide Levels

Polypeptide levels in serum or plasma (or alternatively, urine, more preferably serum) can be assayed in a number of ways. Immunological techniques that involve antibody binding, such as enzyme linked immunosorbent assay (ELISA) and radioimmunoassay (RIA), are typically employed. Colorimetric assays, where they can be employed, are often quite convenient. Where activity assays are available, the activity of a polypeptide of interest can be assayed directly. The polypeptide biomarkers of the present invention may also be quantified using mass spectrometry methods which are well known in the art.

As discussed above, the concentration of these polypeptide biomarkers, especially including glycosylated polypeptide biomarkers in a biological sample may be evaluated by many methods. A direct assay, such as an immunoassay using antibodies that may recognize a glycosylated polypeptide biomarker, is preferred, but other exemplary assays can involve lectins that assay for carbohydrate moieties or any other fingerprinting technique including qualitative or quantitative carbohydrate composition analysis, chromatography, chemical or electrophoresis or isoelectric focusing tests, among others, or any other methods that detect glycosylation variants of polypeptides and/or antibodies to glycosylated or carbohydrate-variant polypeptides. Such assays are described and known in the art.

Immunoassays that can be used to detect unglycosylated and glycosylated polypeptides include, but are not limited to, assays employing specific antibodies to the individual polypeptide biomarkers as otherwise described herein and assays employing nonspecifically defined antibodies obtained by injection of a polypeptide marker obtained from the serum of a patient with an ovarian tumor and/or cancer as described herein, into test animals using standard methods. Antibodies to these polypeptides can be generated by standard means as described, for example, in “Antibodies: A Laboratory Manual” by Harlow and Lane (Cold Spring Harbor Press, 1988), which is hereby incorporated by reference.

For example, a monoclonal anti-polypeptide antibody can be generated by immunizing a mouse with an isolated or recombinant polypeptide (glycosylated or non-glycosylated) pre-treated with neuraminidase, or a cell expressing recombinant the polypeptide pre-treated with neuraminidase. Once an immune response is detected, e.g., antibodies specific for that polypeptide are detected in the mouse serum, the mouse spleen is harvested and splenocytes are isolated. The splenocytes are then fused by well-known techniques to any suitable myeloma cells, for example, cells from cell line SP20 available from the American Type Culture Collection (ATCC). Hybridomas are selected and cloned by limited dilution. The hybridoma clones are then assayed by methods known in the art for cells that secrete antibodies capable of binding the relevant biomarker polypeptide. Ascites fluid, which generally contains high levels of antibodies, can be generated by immunizing mice with positive hybridoma clones.

Any type of fusion phage, monoclonal, or polyclonal antibodies can be used in immunoassays of the invention, so long as the antibodies can be used in a reproducible fashion to identify and quantify polypeptide biomarkers in normal and variant (with cancer) populations.

In one embodiment, an amount of a polypeptide marker (glycosylated or non-glycosylated) can be measured using a capture antibody followed by a labeled secondary antibody using a strategy which is well known in the art. In providing this assay, a first antibody may bind to a polypeptide with polypeptide specificity and a second lableled antibody is used to quantify the target polypeptide. The label on the secondary antibody can comprise any chemical, radioactive, lanthanide, colored dye, or genetic tag used in enzyme-linked immunosorbent assays (ELISAs), Western blots, and other sensitive and specific immunoassays and immunoradiometric assays using known methodology. These include conjugating the antibody with horseradish peroxidase or alkaline phosphatase that are easily measurable, typically using colorimetric, fluorometric or luminescent substrates. Genetic labels include firefly luciferase, employed because luciferase produces a bioluminescent molecule when incubated with its substrate, luciferin.

In other embodiments, polypeptide-specific antibody may be used as a capture antibody, and an antibody specific to a glycosylated or carbohydrate-variant of the polypeptide and/or an abnormal carbohydrate portion thereof can be used as the secondary labeled antibody in an immunoassay such as those described above. Competitive immunoassays employing antibodies to specific glycosylated polypeptide markers can also be employed to detect and quantify the glycosylated polypeptides.

Alternate embodiments using Phytohemagglutinin-L or other carbohydrate-specific lectin can be used in place of the capture antibody or labeled antibody. Alternatively, prior to an immunoassay, a lectin or chromatographic method can be used to extract a glycosylated polypeptide biomarker from a biological sample (serum) or from a fraction that was separated or pooled according to another characteristic, such as pI range (isoelectric focusing). These methods are all well known in the art.

Carbohydrate analyses include qualitative observations of differences in physical properties between non-glycosylated and glycosylated proteins, carbohydrate identification using plant lectins specific to the variant carbohydrate portion of the biomarker (where relevant) by standard lectin screening methods, or any other fingerprinting technique including qualitative or quantitative carbohydrate composition analyses.

Antibody purification may be performed, though separation of protein from others, and evaluation of specific bands or peaks on protein separation may provide the same results. Thus, e.g., mass spectroscopy of a protein sample may indicate that quantitation of a particular peak will allow detection of the corresponding gene product. Multidimensional protein separations may provide for quantitation of specific purified entities.

The observed concentration levels of the polypeptide markers of interest are evaluated to determine whether they provide diagnostic or prognostic information for the prediabetic or diabetic condition being analyzed. The evaluation typically involves a comparison between observed polypeptide levels and either a predetermined polypeptide level or threshold value, or a polypeptide level that characterizes a control sample (“predetermined value”). The control sample can be a sample obtained from a normal (i.e., non-diabetic patient with normal glucose tolerance and no observable insulin resistance) or it can be a sample obtained from a patient with a known condition or disease state. For example, if a cytogenic classification is desired, the biological sample can be interrogated for the expression level of a polypeptide correlated with the cytogenic abnormality, then compared with the serum concentration level of the same polypeptide(s) in a patient known to have the cytogenetic abnormality (or an average expression level for the polypeptide biomarker that characterizes that population).

The present study provides specific identification of multiple polypeptide biomarkers whose expression levels in biological samples will serve as markers to evaluate prediabetes and diabetes cases. These markers are selected for statistical correlation to disease outcome data based on a large number of metabolic syndrome (prediabetic) and diabetic patients.

### Treatment of Metabolic Syndrome (Prediabetes) and Diabetes

The polypeptide biomarkers identified herein may be associated with outcome of a disease state and may provide insight into a treatment regimen. That regimen may be that traditionally used for the treatment of a metabolic syndrome (prediabetic condition) or diabetes (as discussed hereinabove) in the case where the analysis of polypeptide biomarkers from samples taken from the patient predicts a favorable therapeutic outcome, or alternatively, the chosen regimen may be a more aggressive approach (higher dosages of traditional therapies for longer periods of time) or even experimental therapies in instances where the predictive outcome is that of failure of therapy.

In addition, the present invention may provide new treatment methods, agents and regimens for the treatment of diabetes. The polypeptide biomarkers identified herein that are associated with outcome and/or specific disease subtypes or karyotypes are likely to have a specific role in the disease condition, and hence represent novel therapeutic targets. Thus, another aspect of the invention involves treating diabetes by modulating the expression of one or more polypeptides described herein to a desired concentration level.

In the case of those polypeptide biomarkers/gene products (Table 1A) whose increased (upregulation) or decreased (downregulation) expression (whether above or below a predetermined value) is associated with a favorable outcome or failure, the treatment method of the invention will involve enhancing the expression of those gene products in which a favorable therapeutic outcome is predicted by such enhancement and inhibiting the expression of those gene products in which enhanced expression is associated with failed therapy.

Thus, in the case of the polypeptide markers from Table 1A, the increased or decreased expression levels for a particular gene as indicated in the table may become a therapeutic goal in the treatment of diabetes, depending on how the gene producing the polypeptide biomarker responds during diabetes therapy. Therapeutic agents for affecting the increased or decreased expression levels may be identified and used as alternative therapies to traditional treatment modalities for diabetes, especially as the increased or decreased expression of each of these genes becomes a therapeutic goal for the treatment of cancer or the development of agents for the treatment of diabetes. Thus, in this aspect of the present invention, the treatment method of the invention involves enhancing or inhibiting one or more of the polypeptide biomarkers gene or other gene products as such are consistent with a therapeutic outcome.

For a number of the gene products identified herein, as above, increased expression is correlated with positive outcomes in diabetes patients—in others, reduced expression. Thus, the invention includes a method for treating diabetes, that involves administering to a patient a therapeutic agent that causes an increase or decrease in the amount or activity of one or more expressed polypeptide biomarkers as otherwise described in Table 1A. Preferably the increase or decrease in amount or activity of the selected gene product is significant, i.e. it is at least about 10%, preferably at least about 25%, more preferably at least about 50% (1.5×), more preferably at least about 100% (2×) and even more preferably at least about 200% (3×) above the expression level observed in the patient prior to treatment.

The therapeutic agent can be a polypeptide having the biological activity of the polypeptide of interest or a biologically active subunit or analog thereof. Alternatively, the therapeutic agent can be a ligand (e.g., a small non-peptide molecule, a peptide, a peptidomimetic compound, an antibody, or the like) that agonizes (i.e., increases) the activity of the polypeptide of interest. For example, the therapeutic agent may function as a receptor or signaling molecule.

Gene therapies can also be used to increase the amount of a polypeptide of interest in a host cell of a patient. Polynucleotides operably encoding the polypeptide of interest can be delivered to a patient either as “naked DNA” or as part of an expression vector. The term vector includes, but is not limited to, plasmid vectors, cosmid vectors, artificial chromosome vectors, or, in some aspects of the invention, viral vectors. Examples of viral vectors include adenovirus, herpes simplex virus (HSV), alphavirus, simian virus 40, picornavirus, vaccinia virus, retrovirus, lentivirus, and adeno-associated virus. Preferably the vector is a plasmid. In some aspects of the invention, a vector is capable of replication in the cell to which it is introduced; in other aspects the vector is not capable of replication. In some preferred aspects of the present invention, the vector is unable to mediate the integration of the vector sequences into the genomic DNA of a cell. An example of a vector that can mediate the integration of the vector sequences into the genomic DNA of a cell is a retroviral vector, in which the integrase mediates integration of the retroviral vector sequences. A vector may also contain transposon sequences that facilitate integration of the coding region into the genomic DNA of a host cell.

Selection of a vector depends upon a variety of desired characteristics in the resulting construct, such as a selection marker, vector replication rate, and the like. An expression vector optionally includes expression control sequences operably linked to the coding sequence such that the coding region is expressed in the cell. The invention is not limited by the use of any particular promoter, and a wide variety is known. Promoters act as regulatory signals that bind RNA polymerase in a cell to initiate transcription of a downstream (3′ direction) operably linked coding sequence. The promoter used in the invention can be a constitutive or an inducible promoter. It can be, but need not be, heterologous with respect to the cell to which it is introduced.

Another option for increasing the expression of a gene product as described in Table 1 wherein higher expression levels are predictive for favorable outcome is to reduce the amount of methylation of the gene. Demethylation agents, therefore, can be used to re-activate the expression of a gene product in cases where methylation of the gene is responsible for reduced gene expression in the patient.

For other polypeptide biomarkers identified herein as being correlated with therapeutic failure or without outcome high expression of the gene may be associated with a negative outcome rather than a positive outcome. In such instances, where the expression levels of these biomarkers are high, the predicted therapeutic outcome in such patients is therapeutic failure for traditional therapies. In such case, more aggressive approaches to traditional therapies and/or experimental therapies may be attempted.

A cell manufactures proteins by first transcribing the DNA of a gene for that protein to produce RNA (transcription). In eukaryotes, this transcript is an unprocessed RNA called precursor RNA that is subsequently processed (e.g. by the removal of introns, splicing, and the like) into messenger RNA (mRNA) and finally translated by ribosomes into the desired protein. This process may be interfered with or inhibited at any point, for example, during transcription, during RNA processing, or during translation. Reduced expression of the gene(s) leads to a decrease or reduction in the activity of the gene product and, in cases where high expression leads to a therapeutic failure, an expected therapeutic success.

The therapeutic method for inhibiting the activity of a polypeptide biomarker whose high expression (table 1) is correlated with negative outcome/therapeutic failure involves the administration of a therapeutic agent to the patient to inhibit the expression of the gene. The therapeutic agent can be a nucleic acid, such as an antisense RNA or DNA, or a catalytic nucleic acid such as a ribozyme, that reduces activity of the gene product of interest by directly binding to a portion of the gene encoding the enzyme (for example, at the coding region, at a regulatory element, or the like) or an RNA transcript of the gene (for example, a precursor RNA or mRNA, at the coding region or at 5′ or 3′ untranslated regions) (see, e.g., Golub et al., U.S. Patent Application Publication No. 2003/0134300, published Jul. 17, 2003). Alternatively, the nucleic acid therapeutic agent can encode a transcript that binds to an endogenous RNA or DNA; or encode an inhibitor of the activity of the polypeptide of interest. It is sufficient that the introduction of the nucleic acid into the cell of the patient is or can be accompanied by a reduction in the amount and/or the activity of the polypeptide of interest. An RNA captamer can also be used to inhibit gene expression. The therapeutic agent may also be protein inhibitor or antagonist, such as small non-peptide molecule such as a drug or a prodrug, a peptide, a peptidomimetic compound, an antibody, a protein or fusion protein, or the like that acts directly on the polypeptide of interest to reduce its activity.

The invention includes a pharmaceutical composition that includes an effective amount of a therapeutic agent as described herein as well as a pharmaceutically acceptable carrier. These therapeutic agents may be agents or inhibitors of selected polypeptide biomarkers (table 1), especially including monoclonal antibodies of same. Therapeutic agents can be administered in any convenient manner including parenteral, subcutaneous, intravenous, intramuscular, intraperitoneal, intranasal, inhalation, transdermal, oral or buccal routes. The dosage administered will be dependent upon the nature of the agent; the age, health, and weight of the recipient; the kind of concurrent treatment, if any; frequency of treatment; and the effect desired. A therapeutic agent identified herein can be administered in combination with any other therapeutic agent(s) such as immunosuppressives, cytotoxic factors and/or cytokine to augment therapy.

The effect of a treatment regimen on metabolic syndrome (prediabetes) or diabetes can be assessed by evaluating, before, during and/or after the treatment, the concentration level of one or more polypeptide biomarkers as described herein. Preferably, the level of polypeptide markers associated with outcome as described above (preferably, favorable outcome, but also, negative outcome), may be monitored over the course of the treatment period. Optionally profiles showing the concentration levels of multiple selected polypeptide biomarkers associated with outcome can be produced at different times during the course of treatment and compared to each other and/or to an expression profile correlated with outcome.

### Screening for Therapeutic Agents

The invention further provides methods for screening to identify agents that modulate expression levels of the polypeptide biomarkers identified herein that are correlated with outcome, risk assessment or classification, cytogenetics or the like. Candidate compounds can be identified by screening chemical libraries according to methods well known to the art of drug discovery and development (see Golub et al., U.S. Patent Application Publication No. 2003/0134300, published Jul. 17, 2003, for a detailed description of a wide variety of screening methods). The screening method of the invention may be carried out in cell culture, for example using adipocyte cell lines that express known levels of the therapeutic target such as a gene product as otherwise described herein (see also Table 1). The cells are contacted with the candidate compound and changes in levels of protein relative to a control culture or predetermined (threshold or control) values based upon a control culture are measured. Alternatively, polypeptide biomarker levels before and after contact with the candidate compound can be measured. Changes above or below a predetermined value indicate that the compound may have therapeutic utility. Structural libraries can be surveyed computationally after identification of a lead drug to achieve rational drug design of even more effective compounds.

The invention further relates to compounds thus identified according to the screening methods of the invention. Such compounds can be used to treat prediabetic conditions and/or diabetes and can be formulated for therapeutic use as described above.

Active analogs, as that term is used herein, include modified polypeptides. Modifications of polypeptides of the invention include chemical and/or enzymatic derivatizations at one or more constituent amino acids, including side chain modifications, backbone modifications, and N- and C-terminal modifications including acetylation, hydroxylation, methylation, amidation, and the attachment of carbohydrate or lipid moieties, cofactors, and the like.

In certain aspects of the present invention, a therapeutic method may rely on an antibody to one or more gene products predictive of outcome, preferably to one or more gene product which otherwise is predictive of a negative outcome, so that the antibody may function as an inhibitor of a gene product. Preferably the antibody is a human or humanized antibody, especially if it is to be used for therapeutic purposes. A human antibody is an antibody having the amino acid sequence of a human immunoglobulin and include antibodies produced by human B cells, or isolated from human sera, human immunoglobulin libraries or from animals transgenic for one or more human immunoglobulins and that do not express endogenous immunoglobulins, as described in U.S. Pat. No. 5,939,598 by Kucherlapati et al., for example. Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. U.S.A., 90:2551-2555 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

Antibodies generated in non-human species can be “humanized” for administration in humans in order to reduce their antigenicity. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Residues from a complementary determining region (CDR) of a human recipient antibody are replaced by residues from a CDR of a non-human species (donor antibody) such as mouse, rat or rabbit having the desired specificity. Optionally, Fv framework residues of the human immunoglobulin are replaced by corresponding non-human residues. See Jones et al., Nature, 321:522-525 (1986); Riechmann et al., Nature, 332:323-327 (1988); and Presta, Curr. Op. Struct. Biol., 2:593-596 (1992). Methods for humanizing non-human antibodies are well known in the art. See Jones et al., Nature, 321:522-525 (1986); Riechmann et al., Nature, 332:323-327 (1988); Verhoeyen et al., Science, 239:1534-1536 (1988); and (U.S. Pat. No. 4,816,567).

In order to more completely define the repertoire of adipocytokines, we report here a detailed proteomic analysis of the secreted proteome of rodent adipocytes (both immortalized mouse and primary rodent derived) using a combination of shotgun proteomics, glycosylation site mapping, and 2D-reverse phase separations that allowed us to assign more than two hundred secreted polypeptides with high confidence. We also demonstrate via quantitative proteomics that many of the secreted proteins that are regulated by classical induction of insulin resistance (hyperglycemia and chronic insulin exposure) are regulated in a similar fashion by elevated O-GlcNAc level-induced insulin resistance.

For mouse, rat, and human adipocytes, two different insulin resistant conditions (either hyperglycemia with chronic insulin exposure or elevation of O-GlcNAc with a pharmacological inhibtor, PUGNAc) were compared to an insulin responsive condition for defining proteins that are differentially secreted. All protein identifications and relative quantification were performed using proteomic methodologies (chromatography and mass spectrometry). All results were confirmed in independent experiments (in quintuplicate for mouse, in triplicate for rat, and in duplicate for human data presented).

While mass spectrometry is used to identify and quantify these relative differences directly from adipocytes in culture, multiple other methods (such as ELISA, RIA, or multiplexing technologies using specific antibodies to the protein of interest) could be used and the source of material being analyzed could be serum or whole blood as adipocytokines are known to enter the bloodstream. While analysis was performed on adipocytes from three different mammalian species it is expected that the homologous protein from any mammalian species (including but not limited to human, rodents, dogs, and cats) will serve the redundant function and be similarly regulated.

Given that the proteins shown here are altered in response to the induction of insulin resistance from metabolic syndrome (which generally precedes and is a hallmark of type II diabetes), it is expected that changes in individual and/or a combination of the identified proteins or processed polypeptides generated from said protein sequences alone or in combination with other documented adipocytokines will not only serve as diagnostic markers for the metabolic syndrome and type II diabetes but are prognostic and diagnostic markers for the development of type II diabetes as well as the plethora of complications associated with diabetes including but not limited to obesity, cardiovascular disease including atherosclerosis and hypertension, pancreatic cancer, retinopathy, renal disease, erectile dysfunction, dental disease, ketoacidosis, depression, and neuropathy. It is also expected that one or multiple of these proteins will serve as likely therapeutic targets for the treatment of said conditions. Furthermore, as many of these markers were secreted by elevation of O-GlcNAc levels which is observed in the diabetic condition and that we have previously demonstrated is sufficient to induce insulin resistance as measured by insulin stimulated glucose uptake in adipocytes, O-GlcNAc levels alone detected by antibodies should be able to be used as both a prognostic and diagnostic biomarker and modulating O-GlcNAc levels should be seen as a potential therapeutic target.

### Examples

### Experimental Procedures

### Tissue Culture

3T3s cell lines: 3T3-L1 and -F442A fibroblasts were maintained and differentiated on 10 cm plates essentially as previously described^{11,25}. Briefly, two days after the cells reached confluence, cells were induced to differentiate with high (4.5 g/ml, 3T3-L1) or low (3T3-F442A) glucose (1.0 g/ml) DMEM containing 10% fetal bovine serum (3T3-L1) or 10% calf serum (3T3-F442A) via addition of 0.5 mM (115 μg/mL) 3-isobutyl-1-methyxanthine (MIX, Sigma), 1 μM (390 ng/mL) dexamethasone (Sigma), and 175 nM (1 μg/mL) recombinant insulin (human, Roche). After 48 hrs (day 2), the medium was replaced with high (3T3-L1) or low (3T3-F442A) glucose DMEM plus 10% FBS and supplemented with 1 μg/mL insulin for an additional 48 hrs. After 96 hrs (day 4), cells were maintained in appropriate medium depending on the condition being examined.

Rat Primary cell culture: inguinal and retroperitoneal fat pads were removed aseptically from male Sprague-Dawley rats (Harlan Industries, Indianapolis, Ind.) and primary preadipocytes were isolated as described in detail previously^{26,27}. After additional washing and centrifugation steps, the preadipocyte pellet was resuspended in plating medium (high glucose DMEM, antibiotics: PS, 10% FBS) and seeded at an average density of 1.35×10⁴ cells/cm² for inguinal and 1.63×10³ cells/cm² for retroperitoneal on 10 cm dishes for the differentiation. Primary cells were maintained in a humidified atmosphere containing 5% CO₂ in air at 37° C. and cultured to confluence, changing the plating medium every 2 days. After 2 days of confluence, referred to as day 0, the medium was replaced with differentiation medium (high glucose DMEM containing antibiotics and 10% FBS, 0.25 mM MIX, 0.1 μM dexamethasone, and 17 nM insulin) and at day 2, an additional 17 nM of insulin was administered with the plating medium for an additional 48 hrs incubation. The cells were maintained in low glucose DMEM containing 10% FBS and treatments (see below) until harvest.

### Cell Treatments and Sample Preparation

On the designated days (day 4 for 3T3-L1, day 5 for 3T3-F442A, and day 9 for rat primary adipocytes), adipocytes were induced with a combined treatment of low or high glucose containing 10% FBS, with or without insulin (100 nM) and PUGNAc (100 μM, TRC Inc.) according to the experimental conditions described in the results. After 24 hrs incubation, the medium was removed and the cells were washed five times with low or high glucose serum-free DMEM without antibiotics and vitamins and incubated with the last rinse for 15 min. After the final wash was removed, serum-free media supplemented with insulin (1 nM) or PUGNAc (100 μM) was added and cells were incubated for 16 hrs.

After incubation, the conditioned media were harvested with extreme care and then centrifuged once at 1,800 rpm at 4° C. for 7 min. The supernatants were filtered using 1 μm syringe filters (PALL). The samples were then collected in centrifuge tubes and centrifuged again at 30,000×G at 4° C. for 30 min. The samples were then transferred to an equilibrated spin column (Centriprep YM-3, Amicon, Millipore) and buffer-exchanged at 2,800 G and 4° C. into 40 mM ammonium bicarbonate (NH₄HCO₃) plus 1 mM DTT and concentrated. The concentrated samples were denatured with 1 M urea, reduced with 10 mM DTT for 1 hr at 56° C., carboxyamidomethylated with 55 mM iodoacetamide in the dark for 45 min, and then digested with 5 μg trypsin (Promega) in 40 mM NH₄HCO₃ overnight at 37° C. After digestion, the peptides were acidified with 200 μl of 1% trifluoroacetic acid (TFA). Desalting was subsequently performed with C18 spin columns (Vydac Silica C18, The Nest Group, Inc.), and the resulting peptides were dried down in a Speed Vac and stored at −20° C. until analyzed. For the subset of samples to be analyzed for N-linked glycosylation, peptides were resuspended in 19 μl ¹⁸O-water and 1 μl of N-glycanase (PNGaseF, Prozyme) and allowed to incubate for 18 hours at 37° C. Peptides were dried back down and resuspended in 50 μl of 40 mM NH₄HCO₃ with 1 μg of trypsin, to remove any possible C-terminal incorporation of ¹⁸O from residual trypsin activity²⁸, for 4 hours and then dried down and stored at −20° C. until analyzed.

### Whole Cell Extracts and Western Blots

Whole cell extracts were prepared and Western blots performed on equal amounts of SDS-PAGE separated proteins using the anti-O-GlcNAc antibody RL-2 and ERK-2 (as a positive control for loading) essentially as previously described²⁹.

### Analysis of Secreted Proteins by RP and RP/RP-LC-MS/MS

The peptides were resuspended with 78 μl of mobile phase A (0.1% formic acid, FA, in water) and 2 μl of mobile phase B (80% acetonitrile, ACN, and 0.1% formic acid in water) and filtered with 0.2 μm filters (Nanosep, PALL). The samples were loaded off-line onto a nanospray tapered capillary column/emitter (360×75×15 μm, PicoFrit, New Objective) self-packed with C18 reverse-phase (RP) resin (8.5 cm, Waters) in a Nitrogen pressure bomb for 10 min at 1,000 p.s.i. (˜5 ul load) and then separated via a 160-min linear gradient of increasing mobile phase B at a flow rate of ˜200 nl/min directly into the mass spectrometer. One-dimensional (RP separation) LC-MS/MS analysis was performed on a Finnigan LTQ mass spectrometer (ThermoFisher, San Jose, Calif.) equipped with a nanoelectrospray ion source. A full MS spectrum was collected (m/z 350-2000) followed by 8 MS/MS spectra following CID (34% normalized collision energy) of the most intense peaks. Dynamic exclusion was set at 2 for 30 seconds exclusion.

Two-dimensional RP/RP-HPLC separation of peptides using an off-line mode was investigated recently and provides increased practical peak capacity and flexibility^{30,31}. For off-line separations, an Agilent 1100 series with a quaternary pump and variable wavelength detector (Semimicro flow cell) was used. 45 μl of the resuspended peptide mixture was loaded onto a reverse-phase column (C18, 2.1×150 mm, 5 μm, GraceVydac) using 5% of mobile phase C (0.1% trifluoroacetic acid, TFA, in water) at a 100 μl/min flow rate for fraction collection. The peptides were eluted in a 55 min linear gradient from 5 to 60% of mobile phase D (80% ACN and 0.085% TFA in water), followed by a 10 min linear gradient from 60 to 95% of mobile phase B and a 5 min hold to flush the column. The eluent was monitored by UV absorbance detection at 215 nm and the fractions were collected every 4 min. Five aliquots (F1: 15-32%, F2: 32-40%, F3: 40-45%, F4: 45-55%, and F5: 55-85% of mobile phase D) of pooled fractions were dried under vacuum. For LC-MS/MS analysis, the peptides were resuspended with 39 μl of mobile phase A and 1 μl of mobile phase B and loaded onto the capillary C18 column in the pressure bomb for 10 min. Resulting peptides were analyzed as described above via LC-MS/MS except that the slope of the gradients were reduced over a 70 minute period in the appropriate region corresponding to the fraction (F1: 4-30%, F2: 9-35%, F3: 15-42%, F4: 20-55%, and F5: 28-85%).

### Data Analysis

The resulting data was searched against the non-redundant mouse (Mus musculus, 11-7-04), rat (Rattus norvegicus, 7-13-05), and combined human (Homo sapiens), mouse, and rat (HMR, released on 7-13-05) database respectively obtained from the National Center for Biotechnology Information (NCBI) using the TurboSequest algorithm (BioWorks 3.1, Thermo Finnigan)^{32,33}, as well as the reverse mouse, rat, and HMR database to estimate the false positive rate (FPR) and the false discovery rate (FDR) of peptide identification (see Table 1). DTA files were generated for spectra with a threshold of 15 ions and a TIC of 3e3. ZSA, correct ion, combion, and ionquest were all applied over a range of MH⁺ 600-4000. The SEQUEST parameters were set to allow 2.2 Da of precursor ion mass tolerance and 0.1 Da of fragment ion tolerance with monoisotopic mass. Only strict tryptic peptides were allowed with up to three missed internal cleavage sites. Dynamic mass increases of 15.99 and 57.02 Da were allowed for oxidized methionine and alkylated cysteine respectively. In the cases where sites of N-linked glycosylation were investigated with PNGaseF and ¹⁸O-water, a dynamic mass increase of 3 Da was allowed for Asn residues³⁴. The results of the SEQUEST search were filtered to establish the FPR and the FDR as summarized in Table 1. The SEQUEST criteria were determined with FDR of less than 0.1% with the mouse database and 0.2% with the rat database for proteins identified by 2 unique peptides. More stringent SEQUEST criteria were established for one peptide assignments with FDR of below 1% with both databases. For the RP-RP analysis, the five SEQUEST directories were combined and then filtered after redundant peptides were removed manually. iProtein was used to inspect mouse data in terms of secretion signals and localization (a program written by Dawei Lin (web-interface in process) that queries and stores localization information for an entire user-defined database by both SignalP (on the world wide web at cbs.dtu.dk/services/SignalP) and PsortII (psort.nibb.ac.jp/form2.html) and then allows filtered Sequest output files to be screened in a single step and exports the localization data with the sequest data in an excel compatible format).

### Results

### Elevation of the O-GlcNAc Modification via the Induction of Insulin Resistance

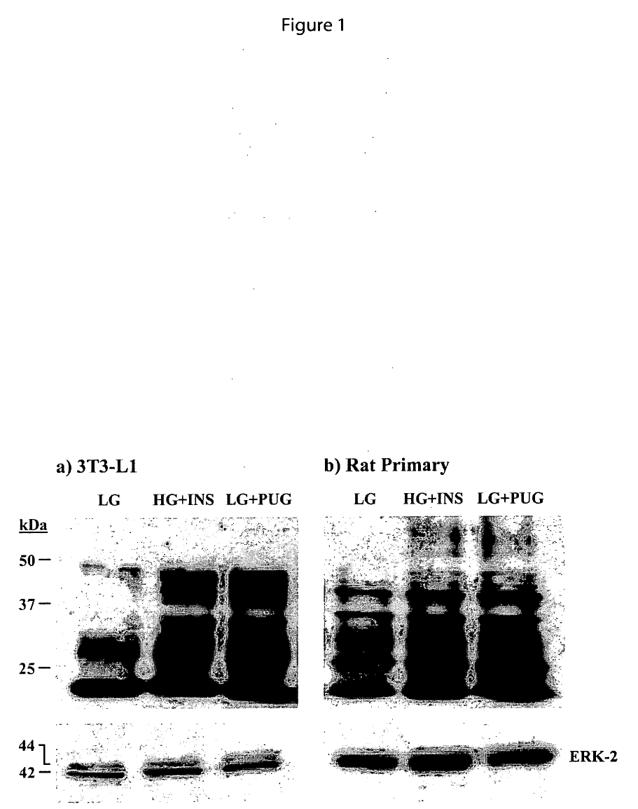

Elevation in O-GlcNAc levels has previously been correlated with insulin resistance and the diabetic condition^{5,8,9,35-40}. Furthermore, induction of insulin resistance in 3T3-L1 adipocytes by hyperglycemia and chronic insulin exposure elevates O-GlcNAc levels¹¹. We have previously shown that elevation of O-GlcNAc levels with the O-GlcNAcase inhibitor PUGNAc induces insulin resistance¹¹. Both insulin resistant conditions (classical hyperglycemia combined with chronic insulin exposure and PUGNAc treatment) elevate O-GlcNAc levels significantly in mouse immortalized and rat primary adipocytes (FIG. 1). The induction of classical insulin resistance has been shown to modulate multiple adipocytokines and O-GlcNAc has been implicated in regulating leptin and adiponectin^{9,16,23,24,41}. The present results illustrate that the induction of either insulin resistant condition elevates O-GlcNAc levels in both immortalized and primary rodent adipocytes.

Analysis of the Secreted Proteome from Differentiated 3T3-L1 and 3T3-F442A Adipocytes

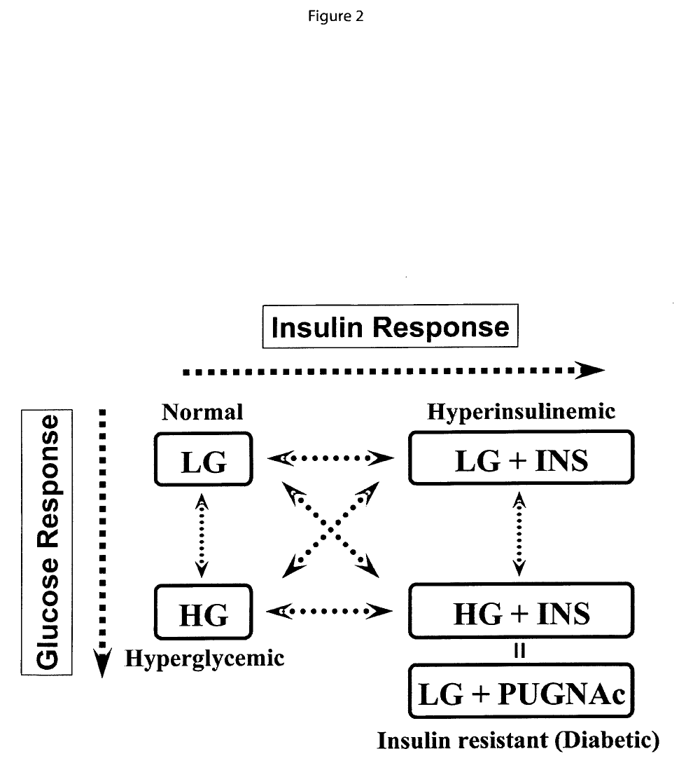

Two immortalized mouse pre-adipocyte cell lines (3T3-L1 and 3T3-F442A) were differentiated into adipocytes using standard methodologies. Adipocytes were then maintained with serum in either physiological glucose (LG, low glucose) in the presence or absence of PUGNAc (PUG) or shifted to hyperglycemic (HG, high glucose) conditions with or without chronic insulin (I/Ins) exposure for 24 hours (FIG. 2). The resulting cells were washed extensively with PBS and identical serum-free medium was replaced onto the cells and cells maintained for an additional 16 hours.

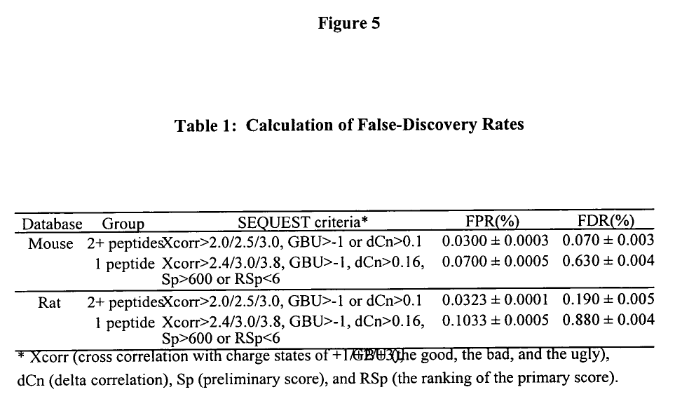

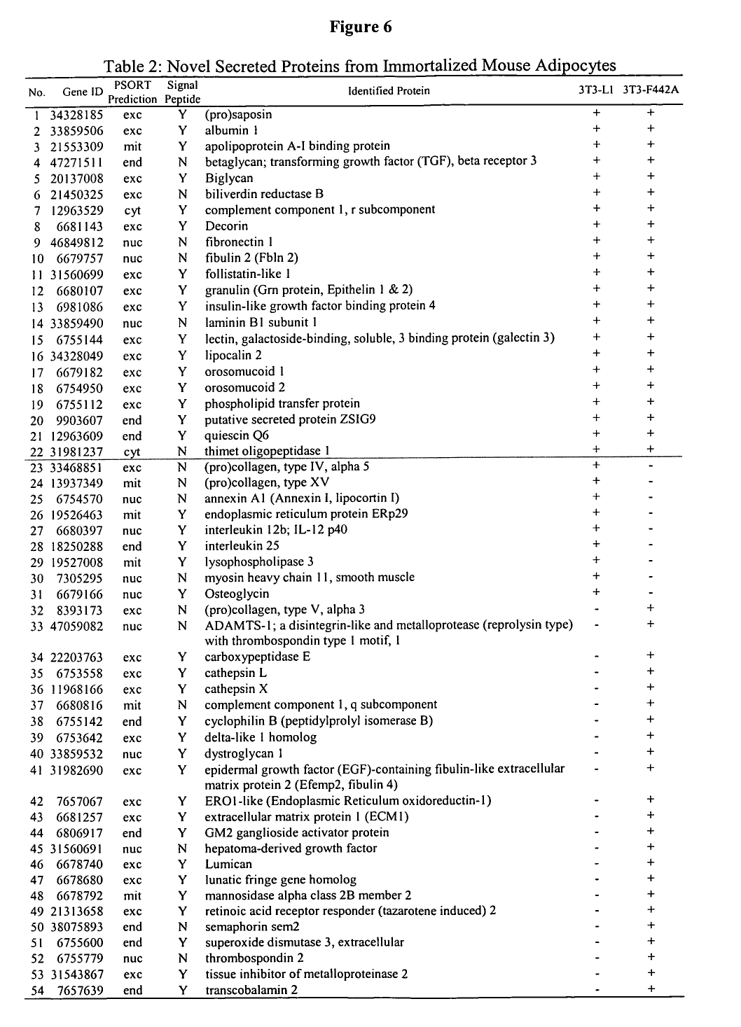

The proteins secreted into the medium during this 16 hour period were collected and processed for analysis by LC-MS/MS. The mass spectra acquired from equivalent normalized aliquots, for quantification by protein coverage, were searched against a mouse non-redundant database using SEQUEST. False-discovery rates were calculated using a reversed database (Table 1, FIG. 5) and all analysis was performed in at least quintuplicate with a new set of cells each time. Each protein identified was assigned a cellular location using PSORT II and SignalP⁴². Because of the large dataset (a total of 50 LC-MS/MS experiments) and the secreted proteome being contaminated by abundant intracellular proteins due presumably to cell lysis, we designed a bioinformatics tool named iProtein that uses pre-calculated databases generated from PSORT II and SignalP for assigning localization (manuscript in preparation). We also validated the secreted proteins using Bioinformatic Harvester (website: harvester.embl.def), the Human Protein Reference Database (using the equivalent human protein when available, at the website: hprd.org/), and the literature¹⁸⁻²⁰. More than half of all the proteins identified appeared to be abundant intracellular proteins suggesting cell lyses during the incubation period. Thus, we performed a shotgun proteomics experiment on cells purposely lysed by scraping in ammonium bicarbonate to identify and easily remove from the secreted proteome list abundant intracellular proteins (data not shown). In total, under all conditions, we assigned 97 proteins as being secreted from 3T3-derived adipocytes. 43 of these proteins had been previously described in other proteomic studies¹⁸⁻²⁰, while 54 proteins were novel in terms of being defined as being secreted from 3T3-derived adipocytes (Table 2, FIG. 6).

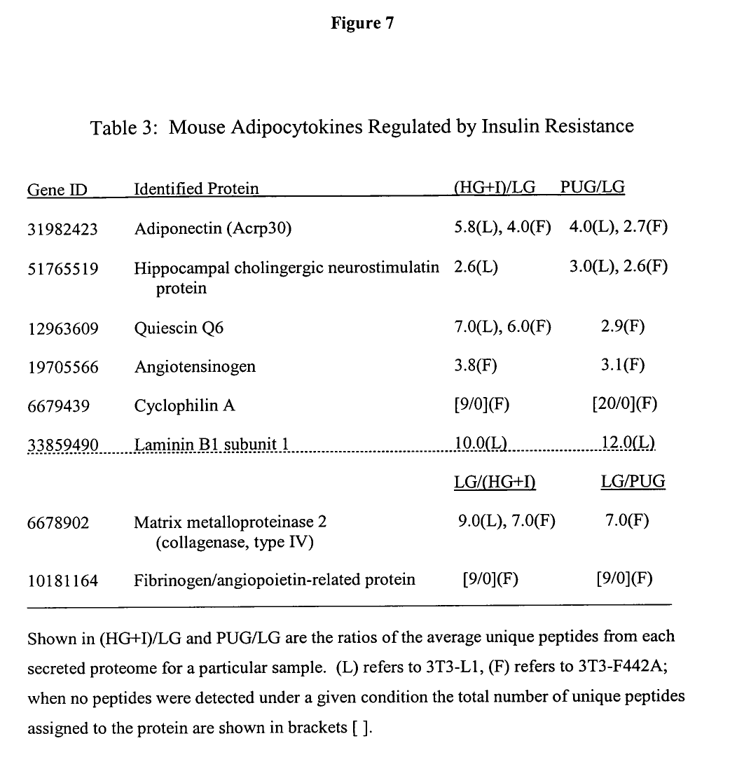

Multiple methodologies exist for protein quantification including isotopic and non-isotopic methods⁴³⁻⁴⁶. To identify proteins that were significantly altered in expression, we identified the total number of unique peptides assigned to each protein in each experiment. An average number of unique peptides were calculated for each protein assigned under the different growth conditions based on the 5 independent experiments under identical conditions. Shown in Table 3 (FIG. 7) are the 8 proteins whose average number of unique peptides changed by at least 2.5-fold between the insulin responsive (LG) versus both of the two different insulin resistance conditions (HG+I and PUG). Also identified were proteins whose abundance only changed under one of the insulin resistant conditions as well as comparisons induced by insulin in low glucose and high glucose alone compared to low glucose conditions.

Quantitative Analysis of the Secreted Proteome from Differentiated Primary Rat Adipocytes by LC-MS/MS and 2D-LC-MS/MS

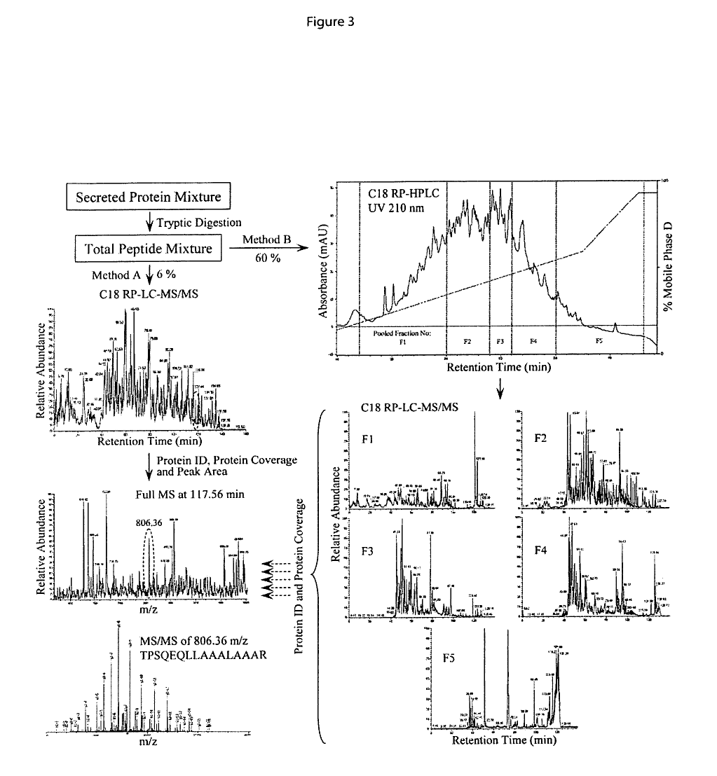

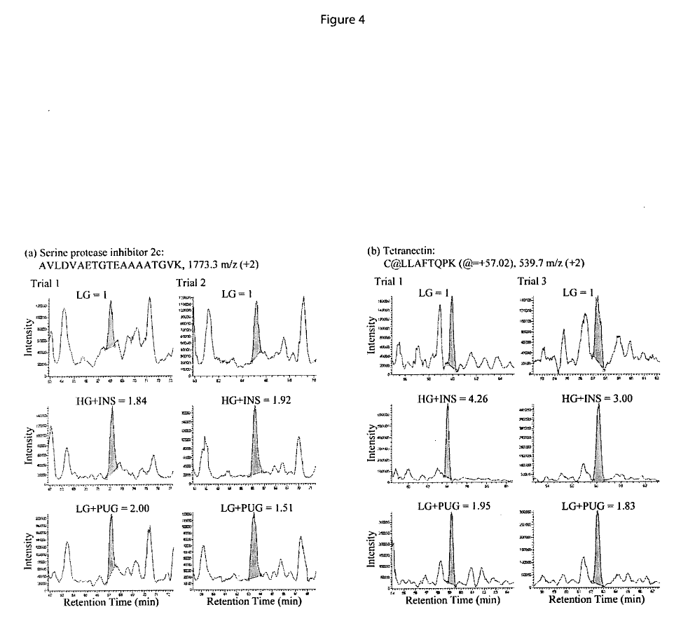

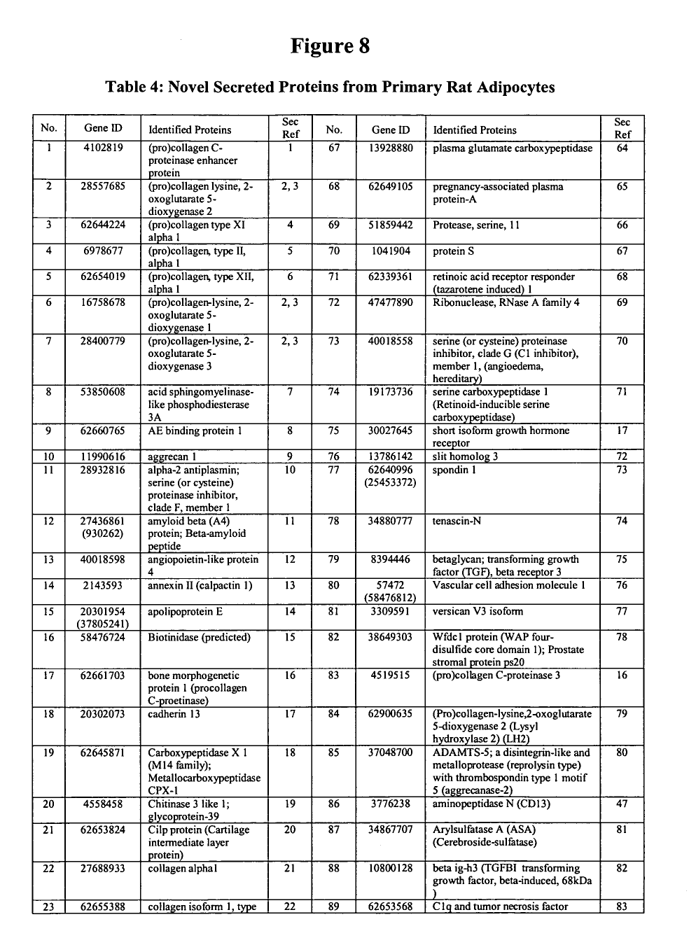

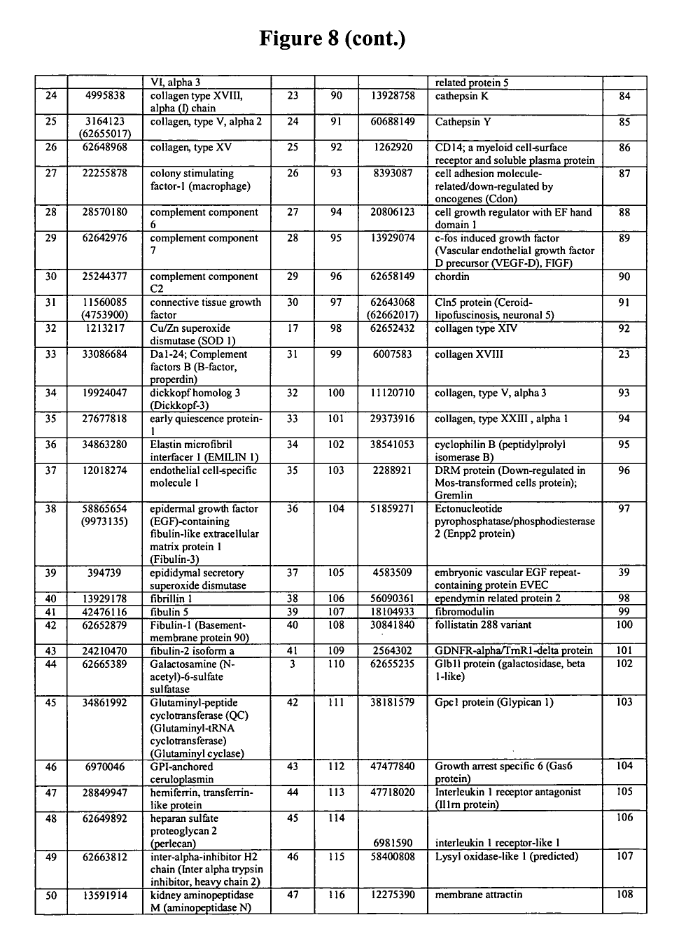

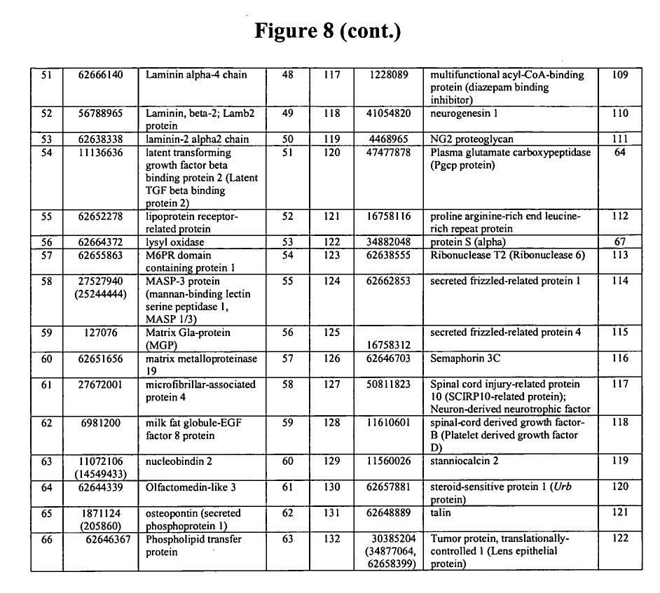

Given that the 3T3 cell lines are an immortalized cell line, have been cultured under high glucose conditions for multiple passages through the years, and that cell lysis was relatively high in these cells, we decided to investigate the secreted proteome of primary rat adipocytes. The secreted proteomes of primary rat adipocytes were harvested under three conditions; two insulin resistance conditions (HG+I and PUG) and one insulin responsive (LG) condition were used. All analysis was carried out on three independently grown and harvested populations for each condition. Once tryptic peptides were generated a fraction of each sample was analyzed by LC-MS/MS essentially as described for the 3T3-derived proteome. The remaining peptides were separated off-line by reverse-phase chromatography and combined into 5 fractions. Each fraction was analyzed by LC-MS/MS with a shallow gradient corresponding to where the peptides eluted in the off-line chromatography. Thus for each independent sample 6 LC-MS/MS experiments were performed (FIG. 3). For each condition performed in triplicate there was 18 LC-MS/MS experiments conducted for a total of 54 LC-MS/MS experiments in the complete set. Combining LC-MS/MS and 2D-LC-MS/MS analyses for all conditions following stringent filtering (see Table 1, FIG. 5) allowed us to assign 203 proteins to the secreted proteome of rat primary adipocytes. Importantly, approximately 75% of the identified proteins in the secreted proteome could be clearly defined as being secreted and not contaminants from cell lysis (compared to 30-35% of the proteins identified in the 3T3-based experiments). Two dimensional analysis increased the total number of unique peptides by slightly more than 2-fold that greatly aided in confident assignments. A total of 132 of these secreted proteins were not identified in previous proteomic screens for secreted proteins from rat primary adipocytes (Table 4, FIG. 8¹⁸).

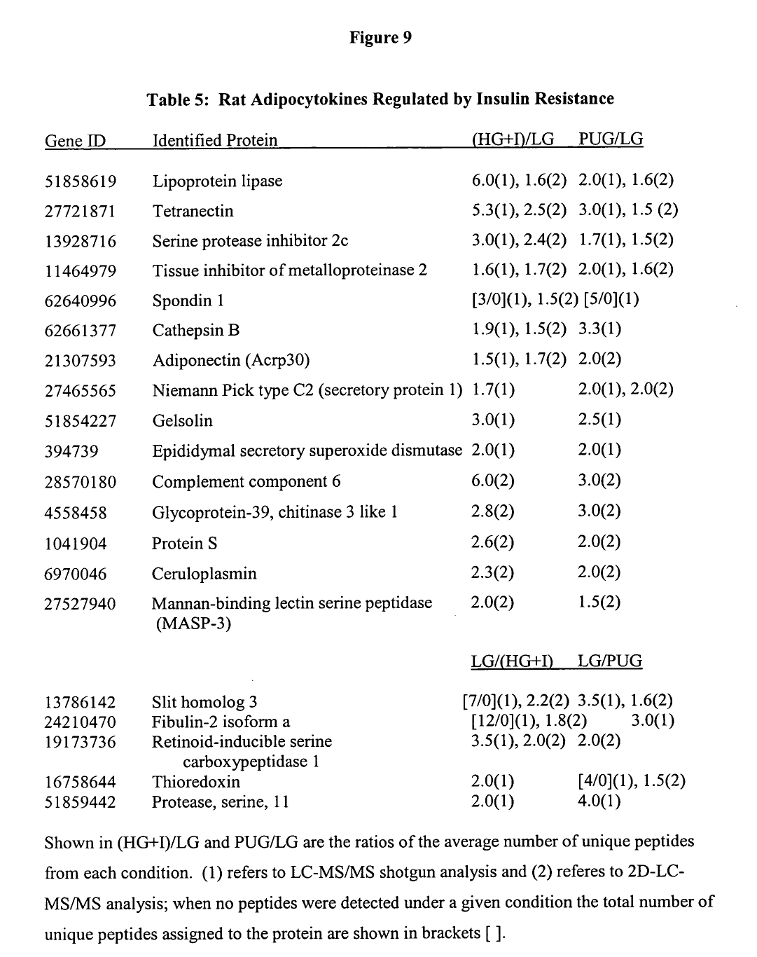

Next, we wanted to compare the secreted proteomes from the two insulin resistant conditions to the insulin responsive conditions. For LC-MS/MS quantitative analysis, we used the number of unique peptides assigned to a protein and area under the peak for normalized reconstructed ion chromatograms of individual peptides that were identified in both conditions. Since we used two different methods for relative quantifications we set our threshold for reporting differences at 150%. For most proteins observed, both methods showed similar trends once the threshold of 1.5 fold change was used. For the two-dimensional analysis, the combined datasets were compared at the level of unique peptides alone to determine proteins showing a change of at least 150%. Reported in table 5 (FIG. 9) are the twenty proteins that changed secretion levels by at least 1.5 fold under both insulin resistant conditions using both LC-MS/MS shotgun data and the two-dimensional data or at least 2 fold change for both conditions under one of the experimental approaches when compared to the insulin responsive condition. Due to the difficulty of assigning unique peptides to specific collagen isoforms (that share high amino acid identity), these proteins are not reported in table 5.

### N-Glycan Site Mapping of the Secreted Proteome of Rodent Adipocytes

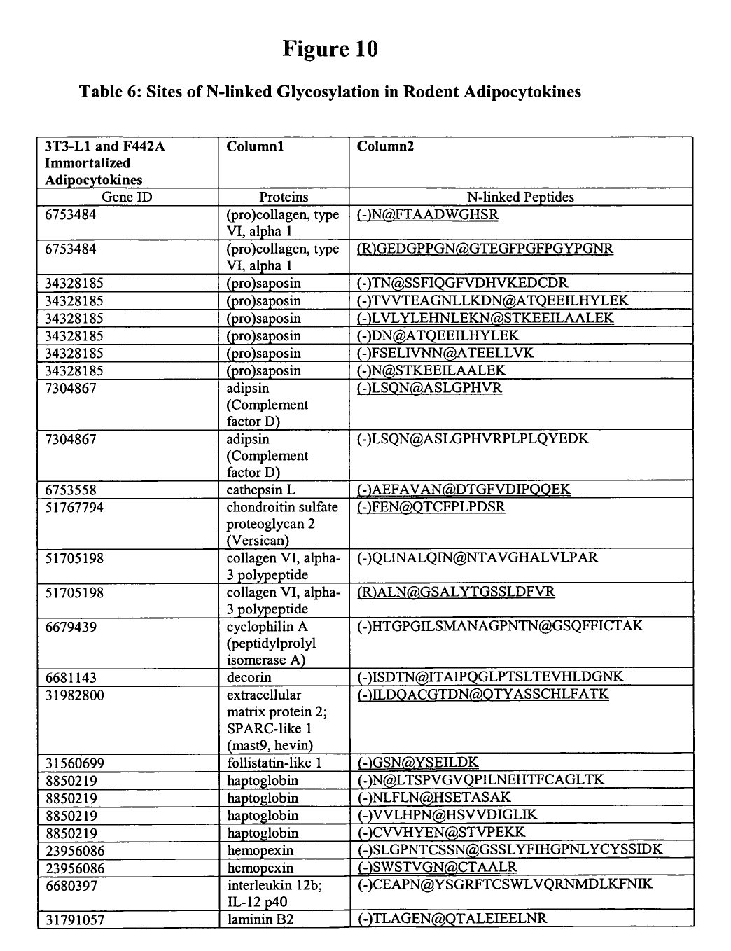

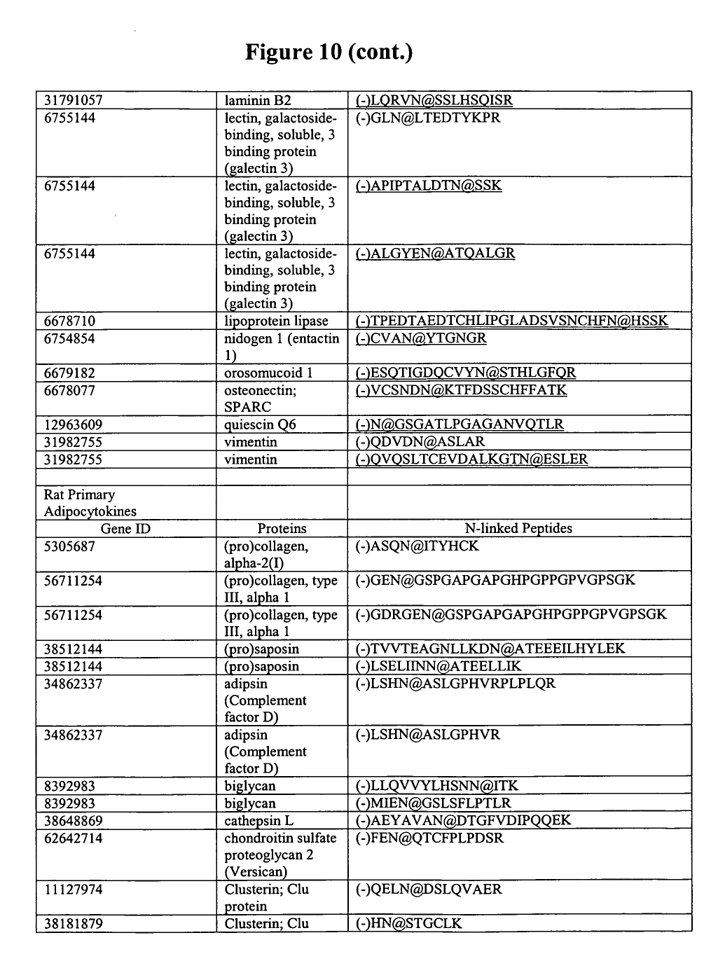

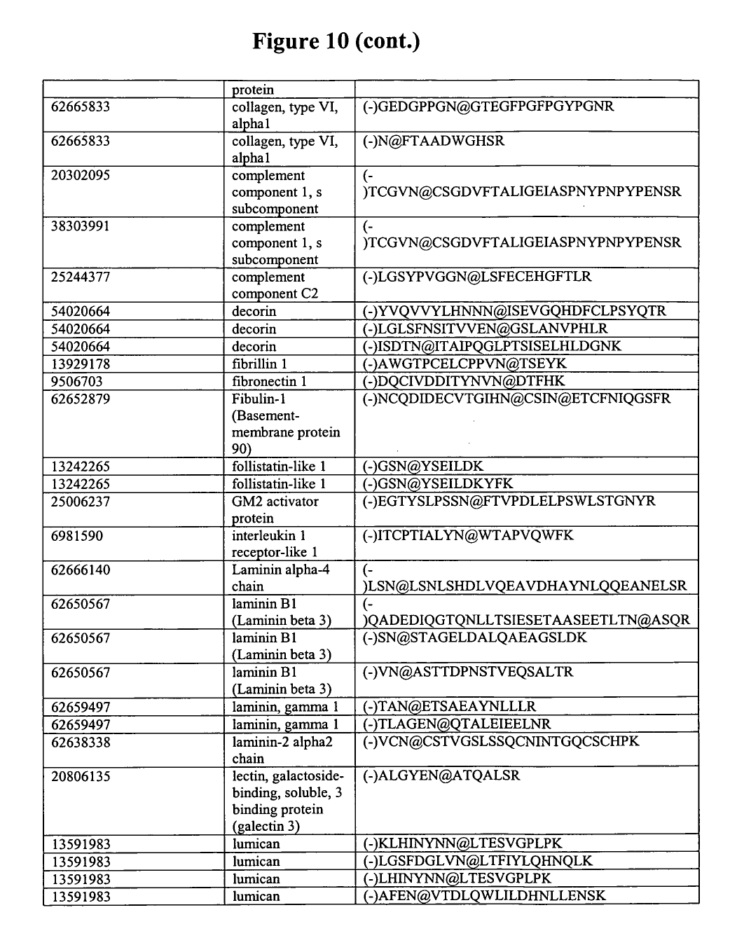

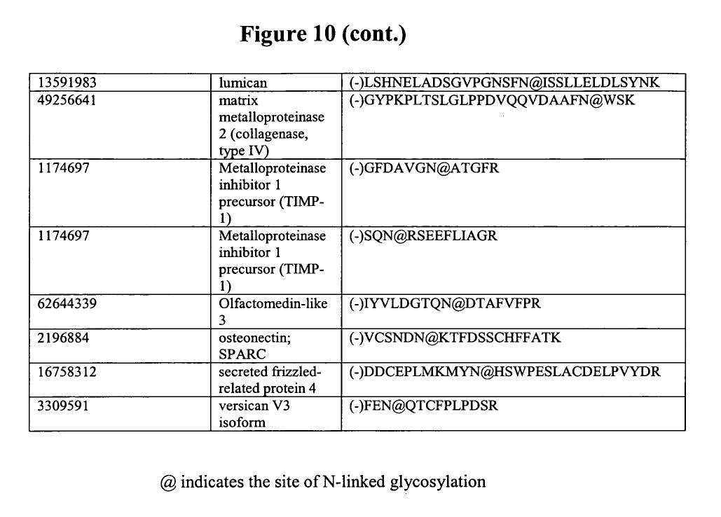

Since the majority of secreted proteins are glycoproteins, one sample from each condition for both mouse and rat adipocytes was treated with PNGaseF in ¹⁸O-water to convert the glycan-modified Asn to an ¹⁸O-Asp residue (mass shift of 3 daltons). These experiments were performed to increase coverage (unique proteins identified in this manner are included in the previous data shown in terms of performing relative quantification) and serve as the first step towards identifying possibly unique glycoforms of these potential biomarkers. In mouse immortalized adipocyte and rat primary adipocyte secreted proteomes, without enrichment, we were able to identify 37 sites on 21 proteins and 48 sites on 30 proteins of N-linked glycosylation, respectively (Table 6, FIG. 10). Care was taken to avoid ¹⁸O incorporation into the C-terminus of peptides by residual trypsin as previously described²⁸. All modified asparagine residues were in the consensus sequence, N-X-S/T, for N-linked glycosylation, adding further proof that our filtering of assignments was sufficiently strict to properly assign peptides and sites of modification.

### Discussion

Using a combination of cell lines and growth conditions, we have more completely defined the secreted proteome of rodent adipocytes. More complete characterization of this secretome should facilitate understanding the central role that adipocytokines play in regulating a variety of processes including energy homeostasis, host defense mechanisms, and insulin sensitivity. Furthermore, this work may aid in the detection and treatment of multiple disease states such as metabolic syndrome, cardiovascular disease, and type II diabetes. Mouse models, especially the adipose-specific Glut4 ablation and overexpression animals, clearly highlight the importance of adipose tissue not only in glucose uptake in that tissue but in whole body insulin action and resistance⁴⁷⁻⁵⁰. The ability of adipose tissue to alter insulin sensitivity in other tissues is likely explained by the endocrine nature of adipocytes. Thus, in an effort to identify potential key signaling molecules, we have begun to explore changes in the secreted proteome of adipocytes when the cells are shifted from being insulin responsive to insulin resistant. Also, given that sugar metabolism is central to diabetes and because we are studying secreted proteins, we have reported preliminary efforts to map some of the N-glycosylation sites but this will need to be focused on in more detail in efforts beyond the scope of this paper to determine if complex glycosylation is being modulated by intracellular glycosylation and/or insulin responsiveness.

Multiple hypotheses have been put forth for the generation of insulin resistance in cells. One model is that cells detect high glucose levels indirectly through generation of UDP-GlcNAc via the hexosamine biosynthetic pathway and down regulate their sensitivity to insulin^{4,39,51,52}. Recent work by our group and others has demonstrated that the mechanism by which UDP-GlcNAc levels impinge on insulin signaling is likely controlled via O-GlcNAc modification of nuclear and cytosolic proteins^{9,11,53}. Given that elevation of O-GlcNAc levels on intracellular proteins via pharmacological inhibition of O-GlcNAcase with PUGNAc alone is sufficient to induce insulin resistance¹¹, provided us with two different means of inducing insulin resistance in rodent adipocytes in culture. Furthermore, leptin and adiponectin have previously been shown to be regulated at the transcriptional level by hexosamine flux, and mice overexpressing the O-GlcNAc transferase in skeletal muscle and adipose tissue display hyperleptinemia^{9,23,24}. Therefore, we decided to focus on secreted proteins that were regulated in a like manner when insulin resistance was produced by either PUGNAc treatment or a combination of hyperglycemia and hyperinsulinemia. Defining this subset of proteins lays the initial groundwork for testing whether any or all of these proteins are potential diagnostic/therapeutic biomarkers for multiple metabolic diseases such as cardiovascular disease and type II diabetes. In fact, several of the proteins we have identified have already been implicated in complications associated with metabolic disease. This data also serves to further illustrate that multiple changes that occur under classical insulin resistance can be recapitulated simply by elevating intracellular glycosylation. Given that elevated O-GlcNAc levels impinge on insulin-dependent glucose uptake and modulate adipocytokine secretion combined with the recent finding that a SNP in OGA (O-GlcNAcase), the neutral, nuclear and cytoplasmic N-acetylglucosaminidase responsible for removing O-GlcNAc from intracellular proteins, correlates strongly with diabetes in Mexican Americans⁵⁴, highlights the importance of understanding this modification as it relates to diabetes. It further demonstrates the potential for modulation of O-GlcNAc levels as a possible therapeutic target in the treatment of insulin resistance-associated diseases such as metabolic syndrome and type II diabetes and their resulting complications.

In the 3T3 system, we identified nine proteins whose steady state levels in the secreted media changed, compared to low glucose, when cells were shifted to either of the insulin resistant conditions. Several of these proteins have previously been associated with Type II diabetes or known complications arising from the induction of insulin resistance. For example, hippocampal cholinergic neurostimulating protein (HCNP), that is upregulated (Table 3), has been shown to play a cardioinhibitory role and cardiovascular disease is the leading cause of death in Type II diabetics⁵⁵. Furthermore, HCNP in the CSF has been associated with Alzheimer's disease and insulin resistance increases the risk of Alzheimer's disease⁵⁶. Further, HCNP has been linked with depression which is a disorder increased in obese and diabetic individuals⁵⁷. Also, angiotensin, that is upregulated (Table 3, FIG. 7), and matrix metalloproteinase 2 (MMP-2) (that is downregulated) have been extensively linked to macro- and microvasular diseases commonly seen in diabetic patients⁵⁸. Further, angiotensin itself has been shown to regulate certain adipocytokines as well as being linked to diabetic retinopathy and nephropathy⁵⁹. Laminins, such as B1 seen in Table 3 (FIG. 7), have been implicated in diabetic nephropathy⁶⁰. In the 3T3 system, as well as the primary adipocytes, an increase in adiponectin was detected. This is consistent with the findings of Hess and colleagues¹⁸, and may indicate that adiponectin, that is known to be suppressed in rodent models of chronic insulin resistance⁶¹, is upregulated initially upon insulin resistance (in an attempt to improve insulin sensitivity?) and later downregulated. Finally, cyclophilin A overexpression, as seen in Table 3, has been linked to pancreatic cancer⁶², and type II diabetes, obesity, and insulin resistance increase the incidence rate of this malignancy⁶³.

Due mainly to the decrease in intracellular contamination and the increased depth of analysis afforded by multidimensional chromatography, we were able to explore the secreted proteome of primary adipocytes in more detail. This is ideal, as unlike 3T3-derived adipocytes, these cells more closely resemble in vivo conditions in that they are not immortal nor have they been passaged under hyperglycemic conditions for long periods of time. Several more insulin resistance-regulated proteins were detected in the rat primary adipocyte secretome. Interestingly, in general, detected protease inhibitors appeared to be upregulated while proteases were downregulated. Similar to the 3T3 cells, we noted several proteins (Table 5, FIG. 9) that were regulated in a similar fashion via the induction of insulin resistance using classical methods (hyperglycemia and chronic insulin) and through the elevation of intracellular glycosylation (PUGNAc treatment). Included on this list of regulated proteins were slit homolog 3 and fibulin-2, both of which were downregulated and have been associated with angiogenesis^{64,65}. We also saw upregulation of lipoprotein lipase which is involved in breaking down bloodstream fat⁶⁶. Multiple proteins involved in oxidative stress and the immune response were also regulated including C6, gelsolin, MASP-3, secretory superoxide dismutates, and thioredoxin. Furthermore, upregulation of proteins involved in extracellular and tissue remodeling such as GP-39, spondin, and protein-S was observed. Interestingly, the cholesterol-binding protein NPC2 was upregulated under both insulin resistance conditions. Finally, upregulation was observed for ceruloplasmin and it has previously been observed that mutations in this gene lead to a diabetic phenotype⁶⁷.

These experiments serve to identify possible prognostic/diagnostic markers for metabolic syndrome (insulin-resistance syndrome), type II diabetes, and complications such as cardiovascular disease resulting from these conditions. Future work is aimed at determining whether steady-state levels of these proteins are altered in the serum of rodent models of diabetes and patients diagnosed with metabolic syndrome, type II diabetes, or resulting complications. Furthermore, given that intracellular elevation of O-GlcNAc levels is modulating the secretion of many of these proteins, the mechanism of action is being investigated. The majority of adipocytokines studied to date are regulated at the level of transcription and O-GlcNAc is known to modify and modulate the activity of a number of transcription factors⁶⁸. Alternatively, O-GlcNAc has been shown to regulate the insulin pathway upstream of AKT and therefore an indirect mode of action may be occurring¹¹. Understanding how perturbations in O-GlcNAc levels, that appear to play a major role in several key aspects of insulin-resistance, are altering the normal processes of adipocytes may provide novel insights into prognostics, diagnostics, and therapeutics for metabolic syndrome, type II diabetes, and the associated complications of these debilitating diseases.

Supporting Information Available: Supporting information referenced herein is available as an excel file. This information is available free of charge at the website: pubs.acs.org. See, “Defining the Regulated Secreted Proteome of Rodent Adipocytes Upon the Induction of Insulin Resistance”, by Jae-Min Lim, et al.

### REFERENCES

1. Brownlee, M., The pathobiology of diabetic complications: a unifying mechanism. Diabetes 2005, 54, (6), 1615-25.

2. Brownlee, M., Biochemistry and molecular cell biology of diabetic complications. Nature 2001, 414, (6865), 813-20.

3. Obici, S.; Rossetti, L., Minireview: nutrient sensing and the regulation of insulin action and energy balance. Endocrinology 2003, 144, (12), 5172-8.

4. Patti, M. E., Nutrient modulation of cellular insulin action. Ann NY Acad Sci 1999, 892, 187-203.

5. McClain, D. A.; Crook, E. D., Hexosamines and insulin resistance. Diabetes 1996, 45, (8), 1003-9.

6. Rossetti, L., Perspective: Hexosamines and nutrient sensing. Endocrinology 2000, 141, (6), 1922-5.

7. Hart, G. W.; Housley, M. P.; Slawson, C., Cycling of O-linked beta-N-acetylglucosamine on nucleocytoplasmic proteins. Nature 2007, 446, (7139), 1017-22.

8. Arias, E. B.; Kim, J.; Cartee, G. D., Prolonged incubation in PUGNAc results in increased protein O-Linked glycosylation and insulin resistance in rat skeletal muscle. Diabetes 2004, 53, (4), 921-30.

9. McClain, D. A.; Lubas, W. A.; Cooksey, R. C.; Hazel, M.; Parker, G. J.; Love, D. C.; Hanover, J. A., Altered glycan-dependent signaling induces insulin resistance and hyperleptinemia. Proc Natl Acad Sci USA 2002, 99, (16), 10695-9.

10. Parker, G. J.; Lund, K. C.; Taylor, R. P.; McClain, D. A., Insulin resistance of glycogen synthase mediated by o-linked N-acetylglucosamine. J Biol Chem 2003, 278, (12), 10022-7.

11. Vosseller, K.; Wells, L.; Lane, M. D.; Hart, G. W., Elevated nucleocytoplasmic glycosylation by O-GlcNAc results in insulin resistance associated with defects in Akt activation in 3T3-L1 adipocytes. Proc Natl Acad Sci USA 2002, 99, (8), 5313-8.

12. Havel, P. J., Update on adipocyte hormones: regulation of energy balance and carbohydrate/lipid metabolism. Diabetes 2004, 53 Suppl 1, S143-51.

13. Stolar, M. W., Insulin resistance, diabetes, and the adipocyte. Am J Health Syst Pharm 2002, 59 Suppl 9, S3-8.

14. Guerre-Millo, M., Adipose tissue hormones. J Endocrinol Invest 2002, 25, (10), 855-61.

15. Guzik, T. J.; Mangalat, D.; Korbut, R., Adipocytokines—novel link between inflammation and vascular function? J Physiol Pharmacol 2006, 57, (4), 505-28.

16. Matsuzawa, Y., The metabolic syndrome and adipocytokines. FEBS Lett 2006, 580, (12), 2917-21.

17. Alvarez-Llamas, G.; Szalowska, E.; de Vries, M. P.; Weening, D.; Landman, K.; Hoek, A.; Wolffenbuttel, B. H.; Roelofsen, H.; Vonk, R. J., Characterization of the human visceral adipose tissue secretome. Mol Cell Proteomics 2007, 6, (4), 589-600.

18. Chen, X.; Cushman, S. W.; Pannell, L. K.; Hess, S., Quantitative proteomic analysis of the secretory proteins from rat adipose cells using a 2D liquid chromatography-MS/MS approach. J Proteome Res 2005, 4, (2), 570-7.

19. Kratchmarova, I.; Kalume, D. E.; Blagoev, B.; Scherer, P. E.; Podtelejnikov, A. V.; Molina, H.; Bickel, P. E.; Andersen, J. S.; Fernandez, M. M.; Bunkenborg, J.; Roepstorff, P.; Kristiansen, K.; Lodish, H. F.; Mann, M.; Pandey, A., A proteomic approach for identification of secreted proteins during the differentiation of 3T3-L1 preadipocytes to adipocytes. Mol Cell Proteomics 2002, 1, (3), 213-22.

20. Wang, P.; Mariman, E.; Keijer, J.; Bouwman, F.; Noben, J. P.; Robben, J.; Renes, J., Profiling of the secreted proteins during 3T3-L1 adipocyte differentiation leads to the identification of novel adipokines. Cell Mol Life Sci 2004, 61, (18), 2405-17.

21. Zvonic, S.; Lefevre, M.; Kilroy, G.; Floyd, Z. E.; DeLany, J. P.; Kheterpal, I.; Gravois, A.; Dow, R.; White, A.; Wu, X.; Gimble, J. M., Secretome of primary cultures of human adipose-derived stem cells: modulation of serpins by adipogenesis. Mol Cell Proteomics 2007, 6, (1), 18-28.

22. Moore, G. B.; Chapman, H.; Holder, J. C.; Lister, C. A.; Piercy, V.; Smith, S. A.; Clapham, J. C., Differential regulation of adipocytokine mRNAs by rosiglitazone in db/db mice. Biochem Biophys Res Commun 2001, 286, (4), 735-41.

23. Hazel, M.; Cooksey, R. C.; Jones, D.; Parker, G.; Neidigh, J. L.; Witherbee, B.; Gulve, E. A.; McClain, D. A., Activation of the hexosamine signaling pathway in adipose tissue results in decreased serum adiponectin and skeletal muscle insulin resistance. Endocrinology 2004, 145, (5), 2118-28.

24. Wang, J.; Liu, R.; Hawkins, M.; Barzilai, N.; Rossetti, L., A nutrient-sensing pathway regulates leptin gene expression in muscle and fat. Nature 1998, 393, (6686), 684-8.

25. Student, A. K.; Hsu, R. Y.; Lane, M. D., Induction of fatty acid synthetase synthesis in differentiating 3T3-L1 preadipocytes. J Biol Chem 1980, 255, (10), 4745-50.

26. Ramsay, T. G.; Hausman, G. J.; Martin, R. J., Pre-adipocyte proliferation and differentiation in response to hormone supplementation of decapitated fetal pig sera. J Anim Sci 1987, 64, (3), 735-44.

27. Wagoner, B.; Hausman, D. B.; Harris, R. B., Direct and indirect effects of leptin on preadipocyte proliferation and differentiation. Am J Physiol Regul Integr Comp Physiol 2006, 290, (6), R1557-64.

28. Angel, P. M.; Lim, J.; Wells, L.; Bergmann, C.; Orlando, R., A potential pitfall in 18-O based N-linked glycosylation site mapping. submitted 2006.

29. Corner, F. I.; Vosseller, K.; Wells, L.; Accavitti, M. A.; Hart, G. W., Characterization of a mouse monoclonal antibody specific for O-linked N-acetylglucosamine. Anal Biochem 2001, 293, (2), 169-77.

30. Gilar, M.; Olivova, P.; Daly, A. E.; Gebler, J. C., Orthogonality of separation in two-dimensional liquid chromatography. Anal Chem 2005, 77, (19), 6426-34.

31. Weatherly, D. B.; Astwood, J. A., 3rd; Minning, T. A.; Cavola, C.; Tarleton, R. L.; Orlando, R., A Heuristic method for assigning a false-discovery rate for protein identifications from Mascot database search results. Mol Cell Proteomics 2005, 4, (6), 762-72.

32. Peng, J.; Elias, J. E.; Thoreen, C. C.; Licklider, L. J.; Gygi, S. P., Evaluation of multidimensional chromatography coupled with tandem mass spectrometry (LC/LC-MS/MS) for large-scale protein analysis: the yeast proteome. J Proteome Res 2003, 2, (1), 43-50.

33. Yates, J. R., 3rd; McCormack, A. L.; Eng, J., Mining genomes with MS. Anal Chem 1996, 68, (17), 534A-540A.

34. Kristiansen, T. Z.; Bunkenborg, J.; Gronborg, M.; Molina, H.; Thuluvath, P. J.; Argani, P.; Goggins, M. G.; Maitra, A.; Pandey, A., A proteomic analysis of human bile. Mol Cell Proteomics 2004, 3, (7), 715-28.

35. Kudlow, J. E., The O-GlcNAcase theory of diabetes: commentary on a candidate gene for diabetes. Mol Genet Metab 2002, 77, (1-2), 1-2.

36. Buse, M. G.; Robinson, K. A.; Marshall, B. A.; Hresko, R. C.; Mueckler, M. M., Enhanced O-GlcNAc protein modification is associated with insulin resistance in GLUT1-overexpressing muscles. Am J Physiol Endocrinol Metab 2002, 283, (2), E241-50.

37. Parker, G.; Taylor, R.; Jones, D.; McClain, D., Hyperglycemia and inhibition of glycogen synthase in streptozotocin-treated mice: role of O-linked N-acetylglucosamine. J Biol Chem 2004, 279, (20), 20636-42.

38. Vosseller, K.; Sakabe, K.; Wells, L.; Hart, G. W., Diverse regulation of protein function by O-GlcNAc: a nuclear and cytoplasmic carbohydrate post-translational modification. Curr Opin Chem Biol 2002, 6, (6), 851-7.

39. Wells, L.; Vosseller, K.; Hart, G. W., A role for N-acetylglucosamine as a nutrient sensor and mediator of insulin resistance. Cell Mol Life Sci 2003, 60, (2), 222-8.

40. Yki-Jarvinen, H.; Virkamaki, A.; Daniels, M. C.; McClain, D.; Gottschalk, W. K., Insulin and glucosamine infusions increase O-linked N-acetyl-glucosamine in skeletal muscle proteins in vivo. Metabolism 1998, 47, (4), 449-55.

41. McClain, D. A.; Alexander, T.; Cooksey, R. C.; Considine, R. V., Hexosamines stimulate leptin production in transgenic mice. Endocrinology 2000, 141, (6), 1999-2002.

42. Chen, Y.; Yu, P.; Luo, J.; Jiang, Y., Secreted protein prediction system combining CJ-SPHMM, TMHMM, and PSORT. Mamm Genome 2003, 14, (12), 859-65.

43. Aebersold, R.; Mann, M., Mass spectrometry-based proteomics. Nature 2003, 422, (6928), 198-207.

44. Gygi, S. P.; Rist, B.; Aebersold, R., Measuring gene expression by quantitative proteome analysis. Curr Opin Biotechnol 2000, 11, (4), 396-401.

45. Old, W. M.; Meyer-Arendt, K.; Aveline-Wolf, L.; Pierce, K. G.; Mendoza, A.; Sevinsky, J. R.; Resing, K. A.; Ahn, N. G., Comparison of label-free methods for quantifying human proteins by shotgun proteomics. Mol Cell Proteomics 2005, 4, (10), 1487-502.

46. Ong, S. E.; Foster, L. J.; Mann, M., Mass spectrometric-based approaches in quantitative proteomics. Methods 2003, 29, (2), 124-30.

47. Abel, E. D.; Peroni, O.; Kim, J. K.; Kim, Y. B.; Boss, O.; Hadro, E.; Minnemann, T.; Shulman, G. I.; Kahn, B. B., Adipose-selective targeting of the GLUT4 gene impairs insulin action in muscle and liver. Nature 2001, 409, (6821), 729-33.

48. Carvalho, E.; Kotani, K.; Peroni, O. D.; Kahn, B. B., Adipose-specific overexpression of GLUT4 reverses insulin resistance and diabetes in mice lacking GLUT4 selectively in muscle. Am J Physiol Endocrinol Metab 2005, 289, (4), E551-61.

49. Postic, C.; Mauvais-Jarvis, F.; Girard, J., Mouse models of insulin resistance and type 2 diabetes. Ann Endocrinol (Paris) 2004, 65, (1), 51-9.

50. Wallberg-Henriksson, H.; Zierath, J. R., GLUT4: a key player regulating glucose homeostasis? Insights from transgenic and knockout mice (review). Mol Membr Biol 2001, 18, (3), 205-11.

51. Marshall, S.; Bacote, V.; Traxinger, R. R., Discovery of a metabolic pathway mediating glucose-induced desensitization of the glucose transport system. Role of hexosamine biosynthesis in the induction of insulin resistance. J Biol Chem 1991, 266, (8), 4706-12.

52. McClain, D. A., Hexosamines as mediators of nutrient sensing and regulation in diabetes. J Diabetes Complications 2002, 16, (1), 72-80.

53. Dias, W. B.; Hart, G. W., O-GlcNAc modification in diabetes and Alzheimer's disease. Mol Biosyst 2007, 3, (11), 766-72.

54. Lehman, D. M.; Fu, D. J.; Freeman, A. B.; Hunt, K. J.; Leach, R. J.; Johnson-Pais, T.; Hamlington, J.; Dyer, T. D.; Arya, R.; Abboud, H.; Goring, H. H.; Duggirala, R.; Blangero, J.; Konrad, R. J.; Stern, M. P., A single nucleotide polymorphism in MGEA5 encoding O-GlcNAc-selective N-acetyl-beta-D glucosaminidase is associated with type 2 diabetes in Mexican Americans. Diabetes 2005, 54, (4), 1214-21.

55. Angelone, T.; Goumon, Y.; Cerra, M. C.; Metz-Boutigue, M. H.; Aunis, D.; Tota, B., The emerging cardioinhibitory role of the hippocampal cholinergic neurostimulating peptide. J Pharmacol Exp Ther 2006, 318, (1), 336-44.

56. Tsugu, Y.; Ojika, K.; Matsukawa, N.; Iwase, T.; Otsuka, Y.; Katada, E.; Mitake, S., High levels of hippocampal cholinergic neurostimulating peptide (HCNP) in the CSF of some patients with Alzheimer's disease. Eur J Neurol 1998, 5, (6), 561-569.

57. Khawaja, X.; Xu, J.; Liang, J. J.; Barrett, J. E., Proteomic analysis of protein changes developing in rat hippocampus after chronic antidepressant treatment: Implications for depressive disorders and future therapies. J Neurosci Res 2004, 75, (4), 451-60.

58. Zaoui, P.; Cantin, J. F.; Alimardani-Bessette, M.; Monier, F.; Halimi, S.; Morel, F.; Cordonnier, D., Role of metalloproteases and inhibitors in the occurrence and progression of diabetic renal lesions. Diabetes Metab 2000, 26 Suppl 4, 25-9.

59. Chaldakov, G. N.; Stankulov, I. S.; Hristova, M.; Ghenev, P. I., Adipobiology of disease: adipokines and adipokine-targeted pharmacology. Curr Pharm Des 2003, 9, (12), 1023-31.

60. Schleicher, E.; Nerlich, A.; Gerbitz, K. D., Pathobiochemical aspects of diabetic nephropathy. Klin Wochenschr 1988, 66, (18), 873-82.

61. Hug, C.; Lodish, H. F., Diabetes, obesity, and Acrp30/adiponectin. Biotechniques 2002, 33, (3), 654, 656, 658 passim.

62. Li, M.; Wang, H.; Li, F.; Fisher, W. E.; Chen, C.; Yao, Q., Effect of cyclophilin A on gene expression in human pancreatic cancer cells. Am J Surg 2005, 190, (5), 739-45.

63. Friedman, G. D.; van den Eeden, S. K., Risk factors for pancreatic cancer: an exploratory study. Int J Epidemiol 1993, 22, (1), 30-7.

64. Fujiwara, M.; Ghazizadeh, M.; Kawanami, O., Potential role of the Slit/Robo signal pathway in angiogenesis. Vasc Med 2006, 11, (2), 115-21.

65. Tsuda, T.; Wang, H.; Timpl, R.; Chu, M. L., Fibulin-2 expression marks transformed mesenchymal cells in developing cardiac valves, aortic arch vessels, and coronary vessels. Dev Dyn 2001, 222, (1), 89-100.

66. Franks, P. W.; Mesa, J. L.; Harding, A. H.; Wareham, N. J., Gene-lifestyle interaction on risk of type 2 diabetes. Nutr Metab Cardiovasc Dis 2007, 17, (2), 104-24.

67. Shang, H. F.; Jiang, X. F.; Burgunder, J. M.; Chen, Q.; Zhou, D., Novel mutation in the ceruloplasmin gene causing a cognitive and movement disorder with diabetes mellitus. Mov Disord 2006, 21, (12), 2217-20.

68. Wells, L.; Whalen, S. A.; Hart, G. W., O-GlcNAc: a regulatory post-translational modification. Biochem Biophys Res Commun 2003, 302, (3), 435-41.

1. A method of diagnosing metabolic syndrome or type 2 diabetes in a patient at risk comprising measuring the concentration of at least one polypeptide marker selected from the group consisting of Lipoprotein lipase, Quiescin Q6, Cathepsin B, Complement Component 6, Hippocampal Cholinergic Neurostimulating Protein (HCNP), Serine Protease Inhibitor 2C, Adiponectin, Angiotensinogen (angiotensin), Cyclophilin A, Laminin B1 subunit 1, cartilage glycoprotein 39, complement factor MASP-3 (MASP-3), Niemann-Pick disease type C2 isoform CRA_b (NPC2), Tetranectin, tissue inhibitor of metalloproteinase 2 (TIMP2), Superoxide dismutase secreted, and Spondin 1, in a biological sample obtained from a patient, and comparing the concentration of the measured polypeptide marker(s) from said patient to a predetermined value, wherein the concentration of said measured polypeptide markers at a level above or below a control is evidence of metabolic syndrome or type 2 diabetes; and diagnosing said patient as having metabolic syndrome or type 2 diabetes.

2. The method according to claim 1 wherein said polypeptide biomarkers comprise at least two of said polypeptide markers.

3. The method according to claim 1 wherein said polypeptide biomarkers comprise at least three of said polypeptide markers.

4. The method according to claim 1 wherein said polypeptide biomarkers comprise at least four of said polypeptide markers.

5. The method according to claim 1 wherein said polypeptide biomarkers comprise at least five of said polypeptide markers.

6. The method according to claim 1 wherein said polypeptide biomarkers comprise at least six of said polypeptide markers.

7. The method according to claim 1 wherein said polypeptide biomarkers comprise at least ten of said polypeptide markers.

8-12. (canceled)

13. The method according to claim 1 where said polypeptide biomarker is at least one biomarker selected from the group consisting of Lipoprotein lipase, Quiescin Q6, Cathepsin B, Complement Component 6, Hippocampal Cholinergic Neurostimulating Protein (HCNP) and Serine Protease Inhibitor 2C.

14. The method according to claim 1 wherein at least four of said biomarkers are measured and a diagnosis of metabolic syndrome or type 2 diabetes is made if the concentration of at least half of said biomarkers is above a control.

15. The method according to claim 1 which diagnoses metabolic syndrome in said patient.

16. The method according to claim 15 which diagnoses type 2 diabetes in said patient.

17. A method of monitoring the therapy for metabolic syndrome or type 2 diabetes in a patient in need thereof comprising obtaining a biological sample from said patient and measuring the expression of one or more polypeptide biomarkers Lipoprotein lipase; Quiescin Q6, Cathepsin B, Complement Component 6, Hippocampal Cholinergic Neurostimulating Protein (HCNP), Serine Protease Inhibitor 2C; Adiponectin, Angiotensinogen (angiotensin), Cyclophilin A, Laminin B1 subunit 1, cartilage glycoprotein 39, complement factor MASP-3 (MASP-3), Niemann-Pick disease type C2 isoform CRA_b (NPC2), Tetranectin, tissue inhibitor of metalloproteinase 2 (TIMP2), Superoxide dismutase secreted, and Spondin 1, wherein a measured concentration of said polypeptide biomarker(s) above or below a control is evidence of effective or favorable therapy.

18-28. (canceled)

29. The method according to claim 17 where said polypeptide biomarker is at least one biomarker selected from the group consisting of Lipoprotein lipase, Quiescin Q6, Cathepsin B, Complement Component 6, Hippocampal Cholinergic Neurostimulating Protein (HCNP) and Serine Protease Inhibitor 2C.

30. The method according to claim 17 wherein at least four of said biomarkers are measured and a diagnosis of metabolic syndrome or type 2 diabetes is made if the concentration of at least half of said biomarkers is above or below a control.

31. A method of determining whether a patient is in remission or cured from metabolic syndrome or type 2 diabetes comprising obtaining a biological sample from said patient and monitoring one or more polypeptide biomarkers selected from the group consisting of Lipoprotein lipase; Quiescin Q6; Cathepsin B; Complement Component 6; Hippocampal Cholinergic Neurostimulating Protein (HCNP); Serine Protease Inhibitor 2C; Adiponectin; Angiotensinogen (angiotensin); Cyclophilin A; Laminin B1 subunit 1; cartilage glycoprotein 39; complement factor MASP-3 (MASP-3); Niemann-Pick disease, type C2, isoform CRA_b (NPC2); Tetranectin; tissue inhibitor of metalloproteinase 2 (TIMP2); Superoxide dismutase secreted; and Spondin 1, wherein an expression at, above or below a control level is evidence of a remission or cure of the condition or disease state.

32. The method according to claim 31 wherein said one or more polypeptide markers is selected from the group consisting of Lipoprotein lipase, Quiescin Q6, Cathepsin B, Complement Component 6, Hippocampal Cholinergic Neurostimulating Protein (HCNP) and Serine Protease Inhibitor 2C.

33. The method according to claim 31 wherein at least four of said biomarkers are measured and a diagnosis of a cure or remission of metabolic syndrome or type 2 diabetes is made if the concentration of at least half of said biomarkers is at, above or below a control.

34. The method according to claim 32 wherein at least four of said biomarkers are measured and a diagnosis of a cure or remission of metabolic syndrome or type 2 diabetes is made if the concentration of at least half of said biomarkers is at or below a control.

35. A method of identifying a potential agent for the treatment of metabolic syndrome or type 2 diabetes comprising exposing adipocytes to a concentration of a drug to be tested and measuring the expression of one or more polypeptide biomarkers selected from the group consisting of Lipoprotein lipase, Quiescin Q6, Cathepsin B, Complement Component 6, Hippocampal Cholinergic Neurostimulating Protein (HCNP), Serine Protease Inhibitor 2C, Adiponectin, Angiotensinogen (angiotensin), Cyclophilin A, Laminin B1 subunit 1, cartilage glycoprotein 39, complement factor MASP-3 (MASP-3), Niemann-Pick disease type C2 isoform CRA_b (NPC2), Tetranectin, tissue inhibitor of metalloproteinase 2 (TIMP2), Superoxide dismutase secreted, and Spondin 1, wherein an expression which is above or below a control sample is evidence that the drug exhibits potential as an agent in the treatment of metabolic syndrome or diabetes.

36. The method according to claim 35 wherein said one or more polypeptide markers is selected from the group consisting of Lipoprotein lipase, Quiescin Q6, Cathepsin B, Complement Component 6, Hippocampal Cholinergic Neurostimulating Protein (HCNP) and Serine Protease Inhibitor 2C.

37. The method according to claim 35 wherein said adipocytes to be exposed to drug are engineered to produce concentrations of more or less of at least one polypeptide biomarker than a control adipocyte cell.

38. The method of claim 1 wherein said biological sample is a blood, serum, plasm or urine sample obtained from said patient.

39. The method according to claim 1 wherein said sample is serum obtained from said patient.

40. The method according to claim 1 wherein said sample is urine obtained from said patient.

41. A method of treating a patient with metabolic syndrome or type 2 diabetes comprising diagnosing metabolic syndrome or type 2 diabetes using the method according to claim 1 and thereafter treating said patient with an exercise regimen, a dietary regimen or a pharmaceutical regimen.

42. A method according to claim 1 wherein a diagnosis of metabolic syndrome or type 2 diabetes in said patient is further used to detect, diagnose and/or treat a condition or disease state selected from the group consisting of obesity, cardiovascular disease including atherosclerosis and hypertension, pancreatic cancer, retinopathy, renal disease, erectile dysfunction, dental disease, ketoacidosis, depression and neuropathy.

43. A diagnostic kit for measuring the serum, plasma or urine concentration of polypeptide biomarkers in a biological sample obtained from a patient to be diagnosed, said kit comprising a sterile vial or receptable for obtaining and/or storing a biological sample from said patient, and an assay comprising at least one antibody which is used to determine the concentration in said sample of at least one polypeptide biomarker selected from the group consisting of Lipoprotein lipase, Quiescin Q6, Cathepsin B, Complement Component 6, Hippocampal Cholinergic Neurostimulating Protein (HCNP), Serine Protease Inhibitor 2C, Adiponectin, Angiotensinogen (angiotensin), Cyclophilin A, Laminin B1 subunit 1, cartilage glycoprotein 39, complement factor MASP-3 (MASP-3), Niemann-Pick disease type C2 isoform CRA_b (NPC2), Tetranectin, tissue inhibitor of metalloproteinase 2 (TIMP2), Superoxide dismutase secreted, and Spondin 1, wherein said concentration in said sample is compared to a control concentration.

44. The kit according to claim 43 wherein said at least one polypeptide biomarker is selected from the group consisting of Lipoprotein lipase, Quiescin Q6, Cathepsin B, Complement Component 6, Hippocampal Cholinergic Neurostimulating Protein (HCNP) and Serine Protease Inhibitor 2C.

45. The kit according to claim 43 wherein said assay is a colorimetric assay.

46. An assay for measuring the concentration in a biological sample obtained from a patient to be diagnosed, of at least one polypeptide biomarker selected from the group consisting of Lipoprotein lipase, Quiescin Q6, Cathepsin B, Complement Component 6, Hippocampal Cholinergic Neurostimulating Protein (HCNP), Serine Protease Inhibitor 2C, Adiponectin, Angiotensinogen (angiotensin), Cyclophilin A, Laminin B1 subunit 1, cartilage glycoprotein 39, complement factor MASP-3 (MASP-3), Niemann-Pick disease type C2 isoform CRA_b (NPC2), Tetranectin, tissue inhibitor of metalloproteinase 2 (TIMP2), Superoxide dismutase secreted, and Spondin 1, said assay comprising at least one antibody which is used to determine the concentration in said sample of said at least one polypeptide biomarker.

47. The assay according to claim 46 which compares said concentration measured with a control concentration in order to facilitate diagnosis of metabolic syndrome or type 2 diabetes in said patient.

48. The assay according to claim 46 which is an ELISA assay.

49. The assay according to claim 46 which is a multiplex assay.

50. The assay according to claim 46 wherein said assay is a colorimetric assay.

51-54. (canceled)
